(19) United States
(12) Patent Application Publication
(10) Pub. No.: US 2026/0309656 A1
(43) Pub. Date: Oct. 8, 2026

(54) GAS SENSOR

(71) Applicant: TDK CORPORATION, Tokyo (JP)

(72) Inventor: Yoshikatsu TANAKA, Tokyo (JP)

(21) Appl. No.: 19/461,906

(22) Filed: Jan. 28, 2026

(30) Foreign Application Priority Data
Feb. 7, 2025 (JP) 2025-019137
Nov. 14, 2025 (JP) 2025-195138

(51) Int. Cl. G01N 25/18; G01N 33/00

(52) U.S. Cl. CPC G01N 25/18; G01N 33/0031

(57) ABSTRACT

Disclosed herein is a gas sensor that includes first and second sensor parts configured to output first and second detection signals, respectively, and a signal processing circuit configured to control the first and second sensor parts such that the signal processing circuit is configured to perform a first gas concentration measurement operation by acquiring the first detection signal, start and repeatedly perform a second gas concentration measurement operation by acquiring the second detection signal when a gas concentration reaches or exceeds a first threshold value, and stop performing the second gas concentration measurement operation when the gas concentration reaches or falls below a second threshold value. An execution frequency per unit time of the second gas concentration measurement operation is higher than an execution frequency per unit time of the first gas concentration measurement operation.

### CROSS-REFERENCE TO RELATED APPLICATION

This application claims the benefit of Japanese Patent Applications No. 2025-019137, filed on February 7, 2025 and No. 2025-195138, filed on November 14, 2025, the entire disclosures of which are incorporated by reference herein.

### BACKGROUND OF THE ART

### Field of the Art

The present disclosure relates to a gas sensor and, more particularly, to a gas sensor capable of measuring the concentration of a target gas whose concentration may undergo rapid and significant changes.

### Description of Related Art

International Publication WO 2020/031517 discloses a gas sensor that measures the concentration of a target gas by heating a detection thermistor and a reference thermistor to different temperatures. In this gas sensor, a dummy heating period is set following a measurement operation, and the heating temperature of the detection thermistor during the measurement period is matched with that of the reference thermistor during the dummy heating period, while the heating temperature of the reference thermistor during the measurement period is matched with that of the detection thermistor during the dummy heating period. This reduces the thermal history difference between the detection thermistor and the reference thermistor.

However, when the frequency of gas concentration measurement operations is low, the measurement results may fail to track rapid and significant changes in the concentration of the target gas. In order for the measurement results to track changes in gas concentration, the frequency of the gas concentration measurement operations may be increased; however, this leads to an undesirable increase in power consumption.

### SUMMARY

A gas sensor according to an aspect of the present disclosure includes: a first sensor part configured to output a first detection signal according to a concentration of a target gas; a second sensor part configured to output a second detection signal according to the concentration of the target gas; and a signal processing circuit configured to control the first and second sensor parts and calculate the concentration of the target gas based on the first and second detection signals. The signal processing circuit is configured to control the first sensor part to repeatedly perform a first gas concentration measurement operation to acquire the first detection signal, control the second sensor part to start and repeatedly perform a second gas concentration measurement operation to acquire the second detection signal in response to the gas concentration calculated from the first detection signal reaching or exceeding a first threshold value, and stop the second gas concentration measurement operation in response to at least one of the gas concentration calculated from the first detection signal and the gas concentration calculated from at least the second detection signal reaching or falling below a second threshold value. An execution frequency per unit time of the second gas concentration measurement operation from its start to its stop is higher than an execution frequency per unit time of the first gas concentration measurement operation during the period in which the first gas concentration measurement operation is repeatedly performed.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 100 according to a first embodiment of the technology described herein;

FIGS. 2A and 2B are schematic views for explaining an example of the gas concentration measurement operation of the sensor parts 11 and 21;

FIG. 3 is a flowchart for explaining an example of the gas concentration measurement operation illustrated in FIG. 2A;

FIGS. 4A and 4B are graphs illustrating examples of changes in the CO₂ concentration in an atmosphere where the CO₂ concentration is controlled, where FIG. 4A illustrates an example in which the first threshold value and the second threshold value are the same, and FIG. 4B illustrates an example in which the first threshold value and the second threshold value are different;

FIG. 5 is a flowchart for explaining an example of the gas concentration measurement operation illustrated in FIG. 2B;

FIG. 6 is a flowchart for explaining the operation of the sensor part 11;

FIG. 7 is a timing chart for explaining the operation of the sensor part 11;

FIG. 8 is a graph illustrating the relationship between the ratio of the off-period Toff2 to the on-period Ton2 (Toff2/Ton2) and the drift per unit time of the CO₂ gas concentration measurement result;

FIG. 9 is a graph illustrating a change in the output signal Vout in an atmosphere where the CO₂ gas concentration is controlled;

FIG. 10 is a timing chart for explaining the operation of the sensor part 11 according to a modification;

FIG. 11 is a flowchart for explaining the operation of the sensor part 21;

FIG. 12 is a timing chart for explaining the operation of the sensor part 21;

FIG. 13 is a timing chart for explaining an example of the operations of the sensor parts 11 and 21 in the operation periods T1 and T2 illustrated in FIG. 2A;

FIG. 14 is a flowchart for explaining a modification of the gas concentration measurement operation illustrated in FIG. 2A;

FIG. 15 is a timing chart for explaining an example of the operations of the sensor parts 11 and 21 in the operation periods T1 and T3 illustrated in FIG. 2B;

FIG. 16 is a flowchart for explaining a first modification of the gas concentration measurement operation illustrated in FIG. 2B;

FIG. 17 is a flowchart for explaining a second modification of the gas concentration measurement operation illustrated in FIG. 2B;

FIG. 18 is a timing diagram to supplement the flowchart shown in FIG. 17;

FIG. 19 is a flowchart for explaining a third modification of the gas concentration measurement operation illustrated in FIG. 2B;

FIG. 20 is a timing diagram to supplement the flowchart shown in FIG. 19;

FIG. 21 is a timing chart for explaining the operation of the sensor part 21 according to a first modification;

FIG. 22 is a flowchart for explaining the operation of the sensor part 21 according to a second modification;

FIG. 23 is a timing chart for explaining an example of the operation of the sensor part 21 according to the second modification;

FIG. 24 is a timing chart for explaining another example of the operation of the sensor part 21 according to the second modification; and

FIG. 25 is a circuit diagram illustrating the configuration of a gas sensor 200 according to a second embodiment of the technology described herein.

### DETAILED DESCRIPTION OF THE EMBODIMENTS

The present disclosure describes a technology enabling measurement results to track rapid and significant changes in the concentration of a target gas while reducing power consumption.

Some embodiments of the present disclosure will be explained below in detail with reference to the accompanying drawings.

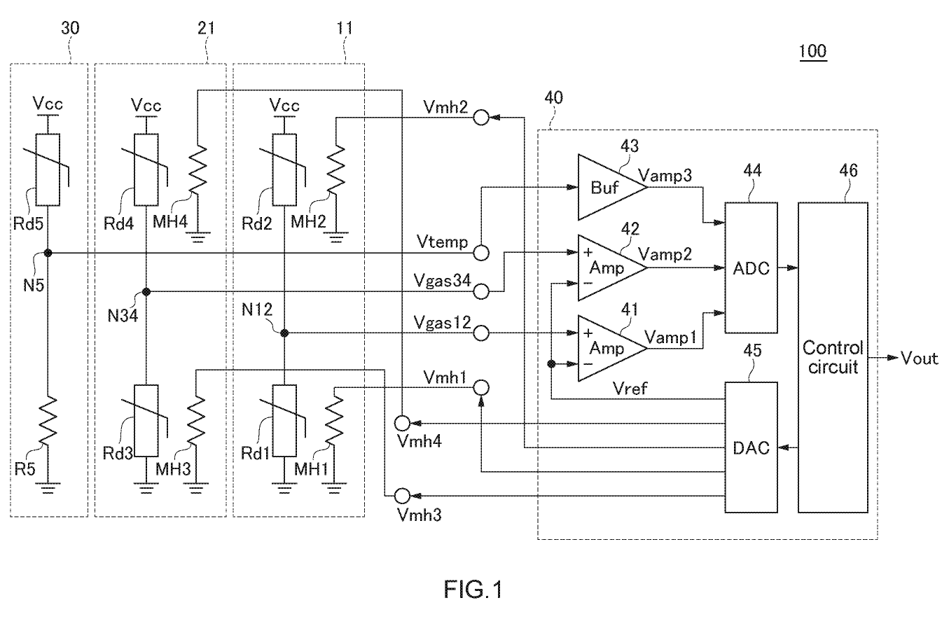

FIG. 1 is a circuit diagram illustrating the configuration of a gas sensor 100 according to a first embodiment of the technology described herein.

As illustrated in FIG. 1, the gas sensor 100 according to the present embodiment includes sensor parts 11 and 21 for detecting the concentration of a target gas, a temperature sensor 30, and a signal processing circuit 40. Although not particularly limited, the gas sensor 100 according to the present embodiment is a heat conduction type gas sensor for detecting the concentrations of CO₂ gas in the measurement atmosphere.

The sensor part 11 includes thermistors Rd2 and Rd1 connected in series in this order between a power supply Vcc and a ground GND and heaters MH1 and MH2 that heat the thermistors Rd1 and Rd2, respectively. A detection signal Vgas 12 output from the sensor part 11 appears at a node N12 between the thermistors Rd1 and Rd2. The thermistor Rd2 is a detection temperature-sensing element, and the thermistor Rd1 is a reference temperature-sensing element. The thermistors Rd1 and Rd2 are resistive elements whose resistance varies in response to changes in temperature. Examples of the material for the thermistors Rd1 and Rd2 and thermistors Rd3, Rd4, and Rd5 to be described later include vanadium oxide, amorphous silicon, polycrystalline silicon, an oxide with a spinel crystal structure containing manganese, titanium oxide, and yttrium-barium-copper oxide. For example, the thermistors Rd1, Rd2, Rd3, Rd4, and Rd5 are NTC thermistors having a negative temperature coefficient of resistance. During the gas concentration measurement, the thermistor Rd1 is heated to, for example, around 300°C (an example of a first temperature range) by the heater MH1, and the thermistor Rd2 is heated to, for example, around 150°C (an example of a second temperature range) by the heater MH2. The first temperature range is a predetermined temperature range included within a range of 250°C or higher and 450°C or lower, for example, a temperature range around 300°C. The second temperature range is a predetermined temperature range included within a range of 100°C or higher and 230°C or lower, for example, a temperature range around 150°C. The term “temperature range” in the present specification refers to a range having, for example, a width of 1°C or lower. For example, the temperature range around 150°C may be defined as a range of 149.5°C or higher and 150.5°C or lower, and the temperature range around 300°C may be defined as a range of 299.5°C or higher and 300.5°C or lower. The thermistor Rd1 is designed to have a predetermined resistance when heated to, for example, 300°C, while the thermistor Rd2 is designed to have a predetermined resistance when heated to, for example, 150°C. The first temperature range (in this example, a temperature range around 300°C) differs from the second temperature range (in this example, a temperature range around 150°C) and is higher than the second temperature range.

When CO₂ gas is present in the measurement atmosphere while the thermistor Rd2, which is the detection temperature-sensing element, is maintained at around 150°C, the heat dissipation characteristics of the thermistor Rd2 change according to the concentration of CO₂ gas. This change appears as a change in the temperature of the thermistor Rd2, i.e., a change in the resistance thereof. For example, CO₂ gas has lower heat dissipation than air, so that the temperature of the thermistor Rd2 rises as the CO₂ gas concentration increases. Here, assume that heating is performed such that the temperature of the thermistor Rd2 becomes 150°C when the CO₂ gas concentration in the measurement atmosphere is, for example, zero. In this case, if CO₂ gas is present in the measurement atmosphere, the temperature of the thermistor Rd2 rises with an increase in the CO₂ gas concentration and exceeds 150°C. As a result, the resistance of the thermistor Rd2 lowers as the CO₂ gas concentration in measurement atmosphere increases.

On the other hand, even when CO₂ gas is present in the measurement atmosphere while the thermistor Rd1, which is the reference temperature-sensing element, is heated to around 300°C, the heat dissipation characteristics of the thermistor Rd1 hardly change according to the CO₂ gas concentration, and the temperature thereof also hardly changes. Accordingly, a CO₂ concentration-dependent change in the resistance of the thermistor Rd1 maintained at around 300°C is sufficiently smaller than a CO₂ concentration-dependent change in the resistance value of the thermistor Rd2 maintained at around 150°C. There may be almost no CO₂ concentration-dependent change in the resistance value of the thermistor Rd1 maintained at around 300°C. As a result, when the thermistors Rd2 and Rd1 are heated to around 150°C and around 300°C, respectively (when heating is performed such that temperatures of the thermistors Rd2 and Rd1 become 150°C and 300°C when the CO₂ gas concentration in the measurement atmosphere is, for example, zero), the detection signal Vgas12 corresponding to the concentration of CO₂ gas in the measurement atmosphere appears at the node N12 between the thermistors Rd1 and Rd2. On the other hand, even when another gas that brings about no significant difference between the heat dissipation characteristics of the thermistor Rd2 exhibited when it is maintained at around 150°C and those of the thermistor Rd1 exhibited when it is maintained at around 300°C is contained in the measurement atmosphere, the concentration of this gas has little influence on the detection signal Vgas12. This allows the sensor part 11 to selectively detect the concentration of CO₂ gas.

The sensor part 21 has the same circuit configuration as that of the sensor part 11. That is, the sensor part 21 includes thermistors Rd4 and Rd3 connected in series in this order between the power supply Vcc and the ground GND and heaters MH3 and MH4 for heating the thermistors Rd3 and Rd4, respectively. A detection signal Vgas34 output from the sensor part 21 appears at a node N34 between the thermistors Rd3 and Rd4. The thermistor Rd4 is a detection temperature- sensing element and may have the same configuration as that of the thermistor Rd2 included in the sensor part 11. The thermistor Rd3 is a reference temperature- sensing element and may have the same configuration as that of the thermistor Rd1 included in the sensor part 11. The thermistors Rd3 and Rd4 are resistive elements whose resistance varies in response to changes in temperature. During the gas concentration measurement, the thermistor Rd3 is heated to, for example, around 300°C (an example of the first temperature range), and the thermistor Rd4 is heated to, for example, around 150°C (an example of the second temperature range). Like the thermistor Rd1 included in the sensor part 11, the thermistor Rd3 is designed to have a predetermined resistance when heated to, for example, 300°C. Further, like the thermistor Rd2 included in the sensor part 11, the thermistor Rd4 is designed to have a predetermined resistance when heated to 150°C.

The temperature sensor 30 includes a thermistor Rd5 and a fixed resistor R5 which are connected in series between the power supply Vcc and the ground GND. A temperature detection signal Vtemp output from the temperature sensor 30 appears at a node N5 between the thermistor Rd5 and the fixed resistor R5. The temperature sensor 30 detects environmental temperature. The environmental temperature is a temperature in the measurement atmosphere. The temperature sensor 30 may be designed so as not to be affected or so as to be hardly affected by heating by, for example, the heaters MH1, MH2, MH3, and MH4.

The signal processing circuit 40 includes differential amplifiers 41 and 42, a buffer 43, an A/D converter (ADC) 44, a D/A converter (DAC) 45, and a control circuit 46.

The differential amplifier 41 compares the detection signal Vgas12 and a reference signal Vref to generate an amplified signal Vamp1 which is a signal obtained by amplifying a difference (= Vgas12 - Vref) in level between the detection signal Vgas12 and the reference signal Vref. The differential amplifier 42 compares the detection signal Vgas34 and the reference signal Vref to generate an amplified signal Vamp2 which is a signal obtained by amplifying a difference (= Vgas34 - Vref) in level between the detection signal Vgas34 and the reference signal Vref. The buffer 43 buffers the temperature detection signal Vtemp to generate an amplified signal Vamp3. The amplified signals Vamp1 to Vamp3 are input to the A/D converter 44. The A/D converter 44 A/D converts the amplified signals Vamp1 to Vamp3 to generate their corresponding digital values and supplies them to the control circuit 46.

The control circuit 46 calculates the concentration of CO₂ gas, which is the target gas, based on the A/D converted amplified signal Vamp1 or Vamp2 and generates an output signal Vout indicating the CO₂ gas concentration. The control circuit 46 calculates the CO₂ gas concentration using a calculation formula set therein. Further, the control circuit 46 supplies digital values of various control parameters to the D/A converter 45. The D/A converter 45 D/A converts the digital values of the various control parameters to generate heater voltages Vmh1 to Vmh4 and the reference signal Vref. The heater voltages Vmh1 to Vmh4 are applied to the heaters MH1 to MH4, respectively, whereby the thermistors Rd1 to Rd4 are heated. The reference signal Vref is supplied to the differential amplifiers 41 and 42.

The control circuit 46 corrects the heater voltages Vmh1 to Vmh4 in accordance with the A/D converted amplified signal Vamp3. For example, the control circuit 46 corrects the heater voltages Vmh1 to Vmh4 such that the temperatures of both the thermistors Rd2 and Rd4 become, for example, 150°C and the temperatures of both the thermistors Rd1 and Rd3 become, for example, 300°C, irrespective of the environmental temperature when the CO₂ gas concentration in the measuring atmosphere is, for example, zero.

The following describes the operation of the gas sensor 100 according to the present embodiment.

The gas sensor 100 according to the present embodiment generates the output signal Vout both from the detection signal Vgas12 of the sensor part 11 (i.e., the amplified signal Vamp1) and from the detection signal Vgas34 of the sensor part 21 (i.e., the amplified signal Vamp2). The signal processing circuit 40 controls the sensor part 11 so as to suppress temporal changes, while it controls the sensor part 21 so as to accurately detect the concentration of the target gas even when the concentration undergoes rapid and significant changes.

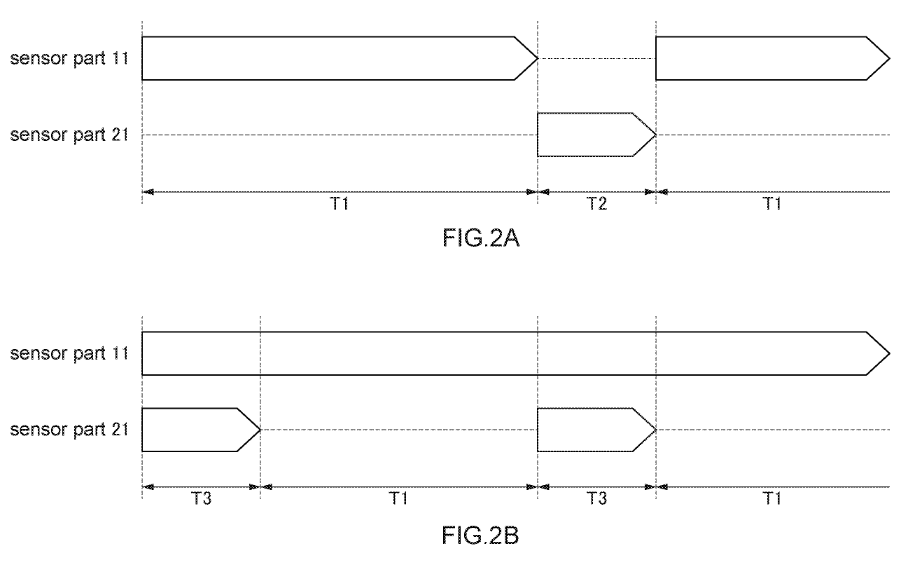

FIGS. 2A and 2B are schematic views for explaining an example of the gas concentration measurement operation of the sensor parts 11 and 21.

In the example illustrated in FIG. 2A, during an operation period T1, the gas concentration measurement operation using the sensor part 11 is performed, whereas the gas concentration measurement operation using the sensor part 21 is stopped. Conversely, during an operation period T2, the gas concentration measurement operation using the sensor part 21 is performed, whereas the gas concentration measurement operation using the sensor part 11 is stopped. The operation period T1 and the operation period T2 are distinct and do not overlap each other. The operation period T2 may be shorter than the operation period T1.

In the example illustrated in FIG. 2B, during an operation period T3, the gas concentration measurement operation using the sensor part 11 and the gas concentration measurement operation using the sensor part 21 are performed in parallel. During the operation period T1, the gas concentration measurement operation using the sensor part 11 is performed, whereas the gas concentration measurement operation using the sensor part 21 is stopped. The operation period T1 and the operation period T3 are distinct and do not overlap each other. The operation period T3 may be shorter than the operation period T1.

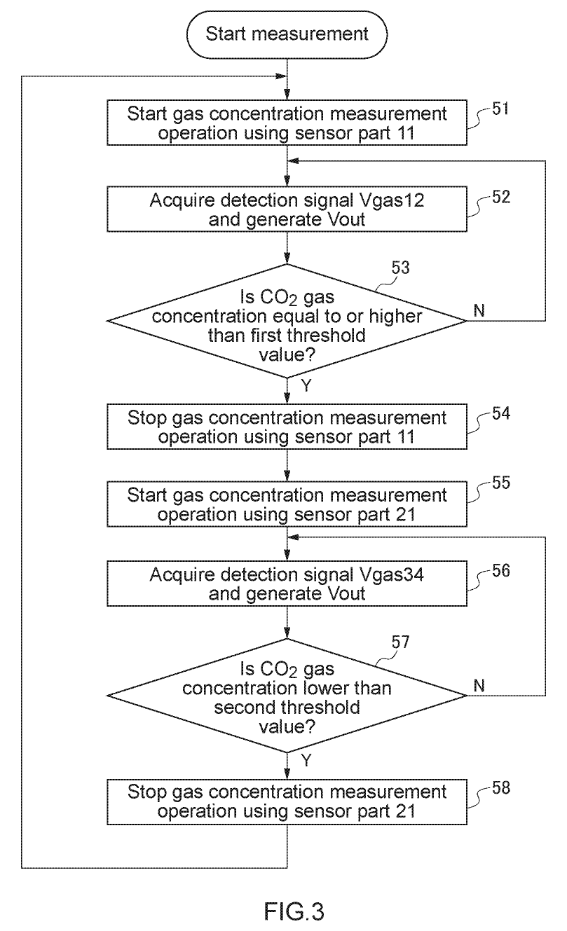

FIG. 3 is a flowchart for explaining an example of the gas concentration measurement operation illustrated in FIG. 2A.

In the example illustrated in FIG. 3, first, the gas concentration measurement operation using the sensor part 11 is started (step 51). When the detection signal Vgas12 appears at the node N12 between the thermistors Rd1 and Rd2 as a result of the gas concentration measurement operation using the sensor part 11, the signal processing circuit 40 acquires the detection signal Vgas12 and generates the output signal Vout based on the acquired detection signal Vgas12 (step 52). Subsequently, the signal processing circuit 40 calculates the CO₂ concentration indicated by the output signal Vout and determines whether the concentration is equal to or higher than a first threshold value (step 53). The CO₂ gas concentration corresponding to the first threshold value is optional and may be, for example, 1000 ppm. When the current CO₂ gas concentration is lower than the first threshold value as a result of the determination, the processing flow returns to step 52, in which the signal processing circuit 40 again performs acquisition of the detection signal Vgas12 and generation of the output signal Vout. On the other hand, when the current CO₂ gas concentration is equal to or higher than the first threshold value, the gas concentration measurement operation using the sensor part 11 is stopped (step 54). During the period from step 51 to step 54, the gas concentration measurement operation using the sensor part 21 is not performed. That is, the period from step 51 to step 54 corresponds to the operation period T1 illustrated in FIG. 2A.

After the gas concentration measurement operation using the sensor part 11 is stopped, the gas concentration measurement operation using the sensor part 21 is started (step 55). When the detection signal Vgas34 appears at the node N34 between the thermistors Rd3 and Rd4 as a result of the gas concentration measurement operation using the sensor part 21, the signal processing circuit 40 acquires the detection signal Vgas34 and generates the output signal Vout based on the acquired detection signal Vgas34 (step 56). Subsequently, the signal processing circuit 40 calculates the CO₂ concentration indicated by the output signal Vout and determines whether the concentration is lower than a second threshold value (step 57). The CO₂ gas concentration corresponding to the second threshold value is optional, and may be the same as or different from the first threshold value (e.g., 1000 ppm). When the current CO₂ gas concentration is equal to or higher than the second threshold value as a result of the determination, the processing flow returns to step 56, in which the signal processing circuit 40 again performs acquisition of the detection signal Vgas34 and generation of the output signal Vout. On the other hand, when the current CO₂ gas concentration is lower than the second threshold value, the gas concentration measurement operation using the sensor part 21 is stopped (step 58), and the processing flow returns to step 51. During the period from step 55 to step 58, the gas concentration measurement operation using the sensor part 11 is not performed. That is, the period from step 55 to step 58 corresponds to the operation period T2 illustrated in FIG. 2A.

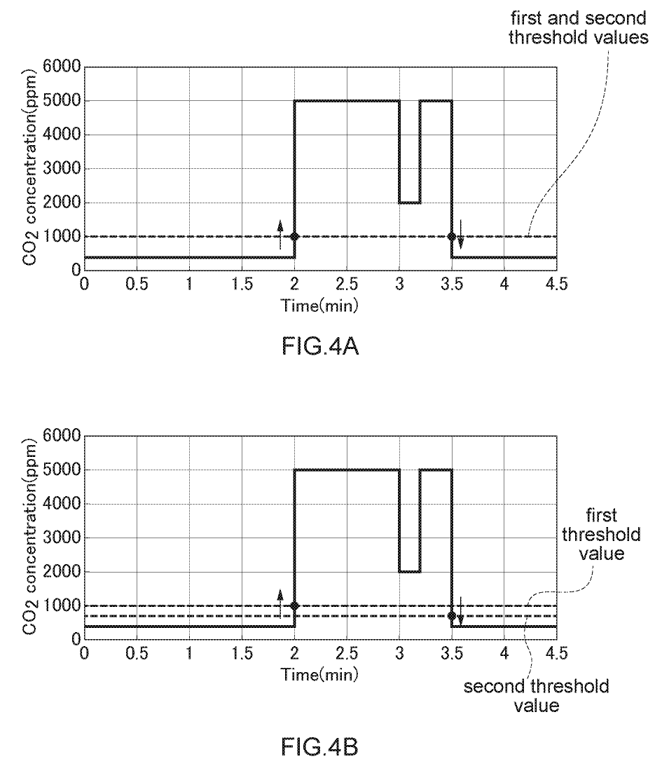

FIGS. 4A and 4B are graphs illustrating examples of changes in the CO₂ concentration in an atmosphere where the CO₂ concentration is controlled. FIG. 4A illustrates an example in which the first threshold value and the second threshold value are the same, whereas FIG. 4B illustrates an example in which the first threshold value and the second threshold value are different.

In both examples illustrated in FIGS. 4A and 4B, the CO₂ gas concentration is maintained constant at 400 ppm during the period before 2 minutes have elapsed from the start. Then, when 2 minutes have elapsed from the start, the CO₂ concentration increases to 5000 ppm, and when 3.5 minutes have elapsed, it returns to 400 ppm. In the example of FIG. 4A, the first and second threshold values are both 1000 ppm. In the example of FIG. 4B, the first threshold value is 1000 ppm, and the second threshold value is 800 ppm.

In these cases, during the period before 2 minutes have elapsed from the start, the gas concentration measurement operation using the sensor part 11 is repeatedly performed since the CO₂ gas concentration is lower than the first threshold value. That is, the period before 2 minutes have elapsed from the start corresponds to the operation period T1 illustrated in FIG. 2A. Then, since the CO₂ gas concentration becomes equal to or higher than the first threshold value when 2 minutes have elapsed from the start, the gas concentration measurement operation using the sensor part 11 is stopped, and the gas concentration measurement operation using the sensor part 21 is repeatedly performed instead. That is, the operation period shifts from T1 to T2 illustrated in FIG. 2A when 2 minutes have elapsed from the start. Further, since the CO₂ gas concentration becomes lower than the second threshold value when 3.5 minutes have elapsed from the start, the gas concentration measurement operation using the sensor part 21 is stopped, and the gas concentration measurement operation using the sensor part 11 is resumed instead. That is, the operation period shifts from T2 to T1 illustrated in FIG. 2A when 3.5 minutes have elapsed from the start.

In the examples of FIGS. 4A and 4B, the CO₂ gas concentration temporarily decreases to 2000 ppm after 2 minutes have elapsed from the start and before 3.5 minutes have elapsed. However, since the concentration does not fall below the second threshold value, the gas concentration measurement operation using the sensor part 21 continues. Further, as illustrated in the example of FIG. 4B, by setting the second threshold value lower than the first threshold value to provide hysteresis, an unstable situation in which the operation periods T1 and T2 switch frequently in a short time can be suppressed.

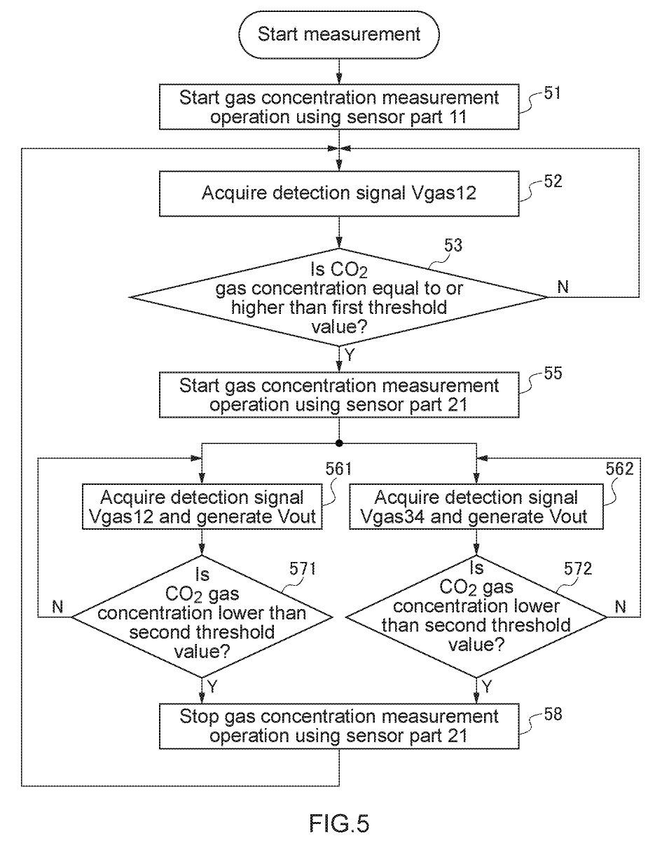

FIG. 5 is a flowchart for explaining an example of the gas concentration measurement operation illustrated in FIG. 2B.

The operation illustrated in FIG. 5 differs from that illustrated in FIG. 3 in the following points: step 54 is omitted; step 56 is replaced with steps 561 and 562; step 57 is replaced with steps 571 and 572; and after the gas concentration measurement operation using the sensor part 21 is stopped in step 58, the processing flow returns to step 52. Other basic operations are the same as those illustrated in FIG. 3, so the same reference numerals are given to the same steps, and overlapping description will be omitted. During the period from step 51 to step 53 illustrated in FIG. 5, the gas concentration measurement operation using the sensor part 21 is not performed. That is, the period from step 51 to step 53 corresponds to the operation period T1 illustrated in FIG. 2B. When the gas concentration measurement operation using the sensor part 21 is started in step 55, the generation of the output signal Vout based on the detection signal Vgas12 (step 561) and the generation of the output signal Vout based on the detection signal Vgas34 (step 562) are performed in parallel. That is, the gas concentration measurement operation using the sensor part 21 is performed without stopping the gas concentration measurement operation using the sensor part 11. Then, it is determined whether the CO₂ gas concentration indicated by the output signal Vout, generated based on the detection signal Vgas12, is lower than the second threshold value (step 571) and it is determined whether the CO₂ gas concentration indicated by the output signal Vout, generated based on the detection signal Vgas34, is lower than the second threshold value (step 572). When the current CO₂ gas concentration is lower than the second threshold value as a result of the determinations in step 571 and/or step 572, the gas concentration measurement operation using the sensor part 21 is stopped (step 58), and the processing flow returns to step 52. The period from step 55 to step 58, including steps 561, 562, 571, and 572, corresponds to the operation period T3 illustrated in FIG. 2B.

In the example illustrated in FIG. 5, the determinations in steps 571 and 572 may be made based on the condition that either both CO₂ concentrations measured using the sensor parts 11 and 21 are lower than the second threshold value, or that at least one of them is lower than the second threshold value.

The following describes the operations of the sensor parts 11 and 21 in more detail.

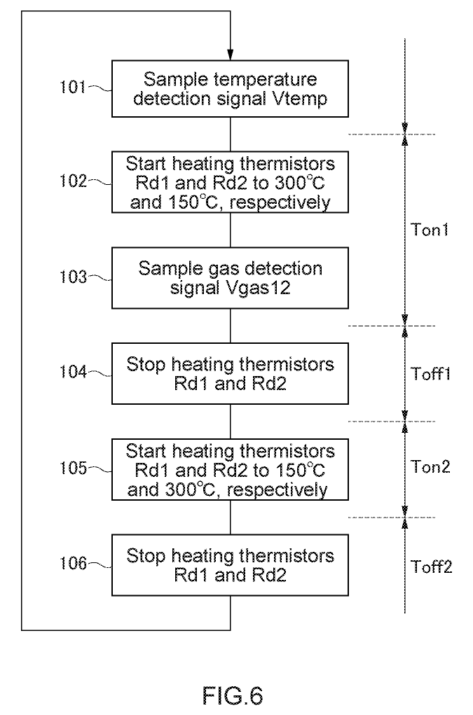

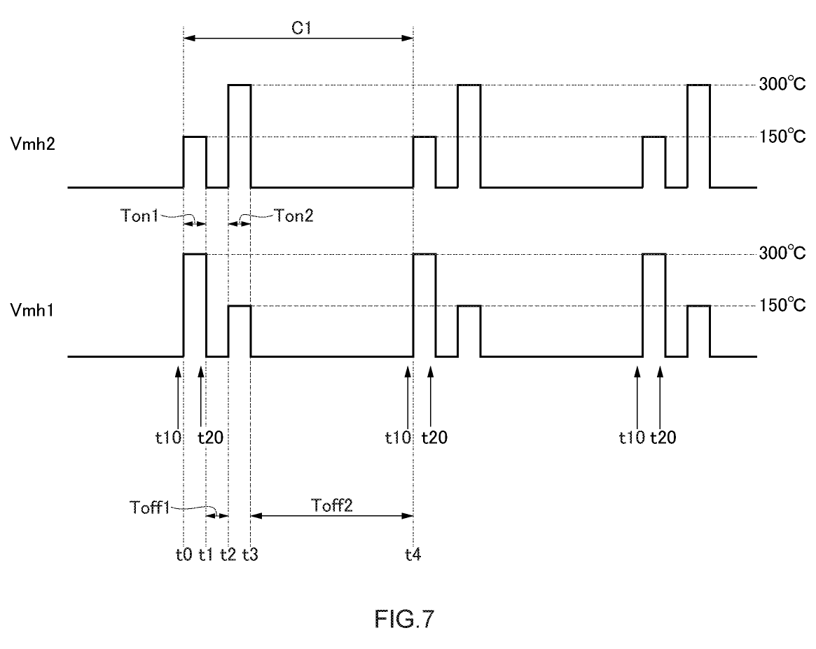

FIG. 6 is a flowchart for explaining the operation of the sensor part 11, and FIG. 7 is a timing chart for explaining the operation of the sensor part 11.

In the gas concentration measurement operation using the sensor part 11, first, the signal processing circuit 40 included in the gas sensor 100 samples the temperature detection signal Vtemp and calculates the ambient temperature (step 101). The sampling of the temperature detection signal Vtemp is performed at timing t10 illustrated in FIG. 7. Timing t10 is immediately before timing t0, at which the thermistors Rd1 and Rd2 start being heated by the heaters MH1 and MH2, respectively.

Then, the control circuit 46 included in the signal processing circuit 40 calculates a heater command value based on the ambient temperature and outputs it to the D/A converter 45 to start heating the thermistors Rd1 and Rd2 (step 102). The heater command value is converted by the D/A converter 45 into heater voltages Vmh1 and Vmh2, which are applied to the heaters MH1 and MH2, respectively. In step 102, the thermistor Rd1 is heated to, for example, about 300°C, and the thermistor Rd2 is heated to, for example, about 150°C. The thermistors Rd1 and Rd2 start being heated at timing t0 illustrated in FIG. 7 (when the concentration of CO₂ gas in the measurement atmosphere is, for example, zero, the thermistor Rd1 is heated to, for example, about 300°C, and the thermistor Rd2 is heated to, for example, about 150°C).

The thermistors Rd1 and Rd2 are not stable in terms of temperature until a predetermined time has elapsed from timing t0 at which they start being heated, so that a predetermined standby time is required until the detection signal Vgas12 is sampled after the start of heating. The signal processing circuit 40 samples the detection signal Vgas12 at timing t20 at which a predetermined standby time has elapsed (step 103). Subsequently, the signal processing circuit 40 calculates the output signal Vout from the detection signal Vgas12 and outputs the calculated output signal Vout to the outside.

Then, the control circuit 46 resets the heater command value to stop heating the thermistors Rd1 and Rd2 (step 104). The thermistors Rd1 and Rd2 stop being heated at timing t1 illustrated in FIG. 7. Thus, the gas concentration measurement operation using the sensor part 11 is completed. In the gas concentration measurement operation using the sensor part 11, the time interval between timing t0 at which the thermistors Rd1 and Rd2 start being heated and timing t1 at which they stop being heated is defined as an on-period Ton1.

After the elapse of a predetermined off-period Toff1 from timing t1, the control circuit 46 outputs the heater command value, calculated based on the ambient temperature, to the D/A converter 45 to start dummy heating for the thermistors Rd1 and Rd2 (step 105). In step 105, the thermistor Rd1 is heated to, for example, about 150°C (an example of the second temperature range), and the thermistor Rd2 is heated to, for example, about 300°C (an example of the first temperature range). The thermistors Rd1 and Rd2 start being heated at timing t2 illustrated in FIG. 7. Thus, the off-period Toff1 is defined as the time interval between timing t1 at which the thermistors Rd1 and Rd2 stop being heated and timing t2 at which the thermistors Rd1 and Rd2 start being heated.

After the elapse of a predetermined on-period Ton2 from timing t2, the control circuit 46 resets the heater command value to stop heating the thermistors Rd1 and Rd2 (step 106). The thermistors Rd1 and Rd2 stop being heated at timing t3 illustrated in FIG. 7. Thus, the dummy heating operation is completed. In the dummy heating operation, the time interval between timing t2 at which the thermistors Rd1 and Rd2 start being heated and timing t3 at which they stop being heated is defined as the on-period Ton2.

After the elapse of a predetermined off-period Toff2 from timing t3, the control circuit 46 resumes the gas concentration measurement operation. For example, the signal processing circuit 40 samples the temperature detection signal Vtemp and calculates the ambient temperature (step 101), and the control circuit 46 outputs the heater command value calculated based on the ambient temperature, to thereby start heating the thermistors Rd1 and Rd2 (step 102). The thermistors Rd1 and Rd2 start being heated at timing t4 illustrated in FIG. 7. Thus, the off-period Toff2 is defined as the time interval between timing t3 at which the thermistors Rd1 and Rd2 stop being heated and timing t4 at which the thermistors Rd1 and Rd2 start being heated again.

By repeatedly performing the above-described operation at predetermined intervals, the concentration of the target gas contained in the surrounding environment can be detected periodically. An execution cycle C1 of the gas concentration measurement operation using the sensor part 11 is defined as the period from time t0 to time t4. The execution cycle C1 may be set so as to reduce the power consumption of the gas sensor 100 in environments where the CO₂ concentration is relatively stable. The execution cycle C1 is set to, for example, 30 seconds.

During on-period Ton1, in which the gas concentration measurement operation is performed, the thermistor Rd1 is heated to about 300°C and the thermistor Rd2 is heated to about 150°C. Conversely, during on-period Ton2, in which the dummy heating operation is performed, the thermistor Rd1 is heated to about 150°C and the thermistor Rd2 is heated to about 300°C. This reduces the thermal history difference between the thermistors Rd1 and Rd2 to suppress temporal changes in the sensor part 11 due to the thermal history difference. In order to further reduce the thermal history difference, the on-periods Ton1 and Ton2 may be set to the same length.

On the other hand, the lengths of the off-period Toff1 and the off-period Toff2 need not be the same, and the off-period Toff2 may be longer than the off-period Toff1. That is, a next gas concentration measurement operation is performed immediately after the off-period Toff2, so that by providing a sufficient length for the off-period Toff2, it is possible to reduce measurement errors due to the influence of residual heat. The influence of residual heat appears as temporal drift in the value of the output signal Vout.

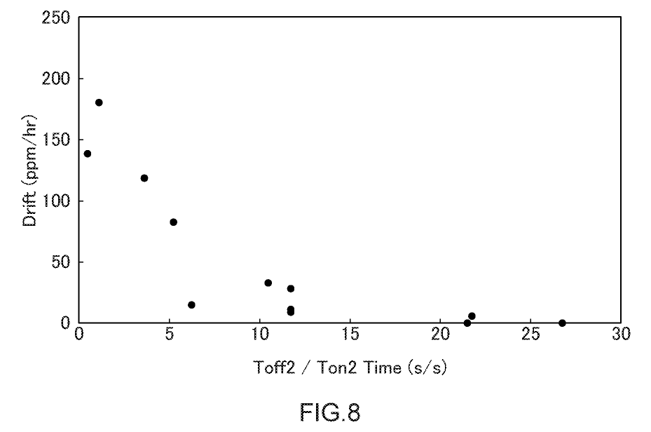

FIG. 8 is a graph illustrating the relationship between the ratio of the off-period Toff2 to the on-period Ton2 (Toff2/Ton2) and the drift per unit time of the CO₂ gas concentration measurement obtained from the output signal Vout in an atmosphere where the CO₂ concentration is maintained constant at 400 ppm.

As can be seen from FIG. 8, the drift amount per unit time decreases as the ratio of the off-period Toff2 to the on-period Ton2 (Toff2/Ton2) becomes larger. The drift amount per unit time is substantially saturated when the ratio of the off-period Toff2 to the on-period Ton2 becomes 10 or greater and becomes substantially zero when the ratio of the off-period Toff2 to the on-period Ton2 becomes 20 or greater. Considering this, in order to sufficiently suppress the drift of the measurement result of the CO₂ gas concentration due to the influence of residual heat, the off-period Toff2 may be made 10 or more times longer and, may be made 20 or more times longer than the on-period Ton2. There is no upper limit to the ratio of the off-period Toff2 to the on-period Ton2. However, an excessively long off-period Toff2 prolongs the time interval between output signal Vout measurements. Therefore, the length of the off-period Toff2 may be set according to the intended purpose.

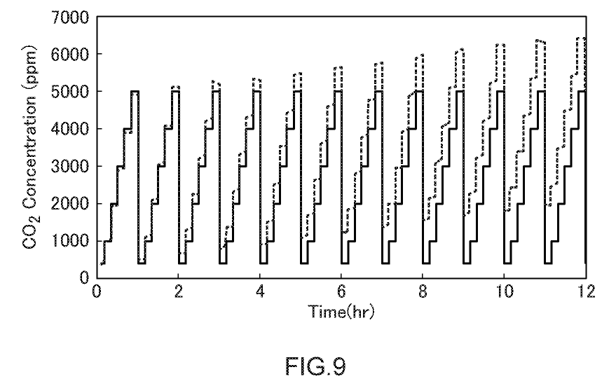

FIG. 9 is a graph illustrating a change in the output signal Vout (measurement result of the CO₂ gas concentration obtained from the output signal Vout) in an atmosphere where the CO₂ gas concentration is controlled. In this graph, the solid line denotes a case where the ratio of the off-period Toff2 to the on-period Ton2 (Toff2/Ton2) is set to 21.5, and the dashed line denotes a case where the ratio of the off-period Toff2 to the on-period Ton2 (Toff2/Ton2) is set to 3.63. The CO₂ gas concentration is changed stepwise (1000 ppm, 2000 ppm. 3000 ppm, 4000 ppm, 5000 ppm) with 400 ppm set as a reference value. The ratio of the off-period Toff1 to the on-period Ton1 (Toff1/Ton1) is set to a fixed value ((Toff1/Ton1)= 5).

As can be seen from FIG. 9, when the value of Toff2/Ton2 is 21.5, the output signal Vout indicates an accurate value, whereas when the value of Toff2/Ton2 is 3.63, the drift occurring in the output signal Vout becomes larger with passage of time.

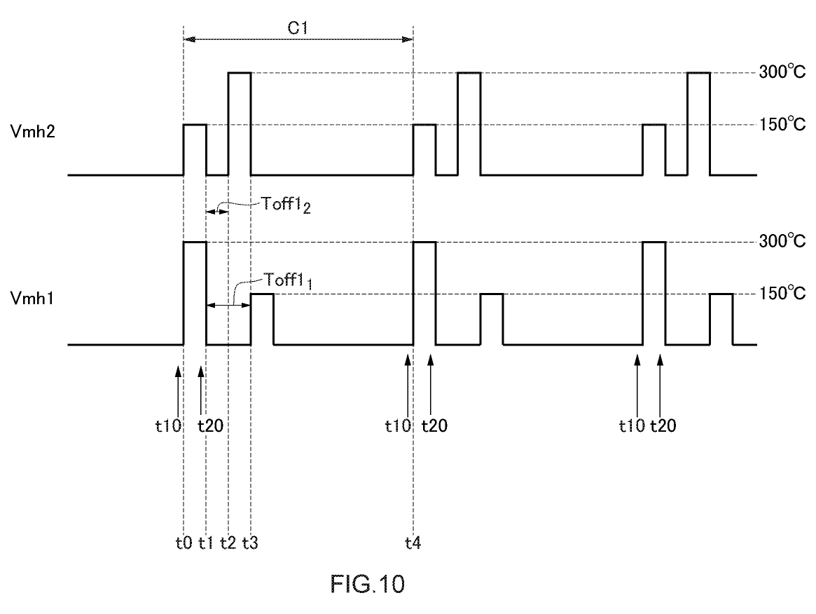

FIG. 10 is a timing chart for explaining the operation of the sensor part 11 according to a modification.

As illustrated in FIG. 10, in the modified operation of the sensor part 11, an off-period Toff1₁ for the thermistor Rd1 is set longer than an off-period Toff1₂ for the thermistor Rd2. The off-period Toff1₁ is time interval between the operation of heating the thermistor Rd1 to, for example, around 300°C (first temperature range) using the heater MH1 in the gas concentration measurement operation and the operation of heating the thermistor Rd1 to, for example, around 150°C (second temperature range) using the heater MH1 in the dummy heating operation. The off-period Toff1₂ is time interval between the operation of heating the thermistor Rd2 to, for example, around 150°C (second temperature range) using the heater MH2 in the gas concentration measurement operation and the operation of heating the thermistor Rd2 to, for example, around 300°C (first temperature range) using the heater MH2 in the dummy heating operation. In the example illustrated in FIG. 10, the off-period Toff1₁ for thermistor Rd1 ends at timing t3. This reduces the temperature difference between the thermistors Rd1 and Rd2 at the start of the dummy heating operation, thereby further reducing the thermal history difference between them. Thus, during the dummy heating operation, the thermistors Rd1 and Rd2 need not be heated simultaneously.

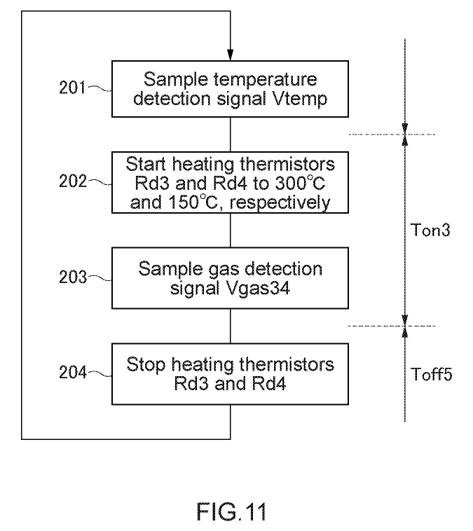

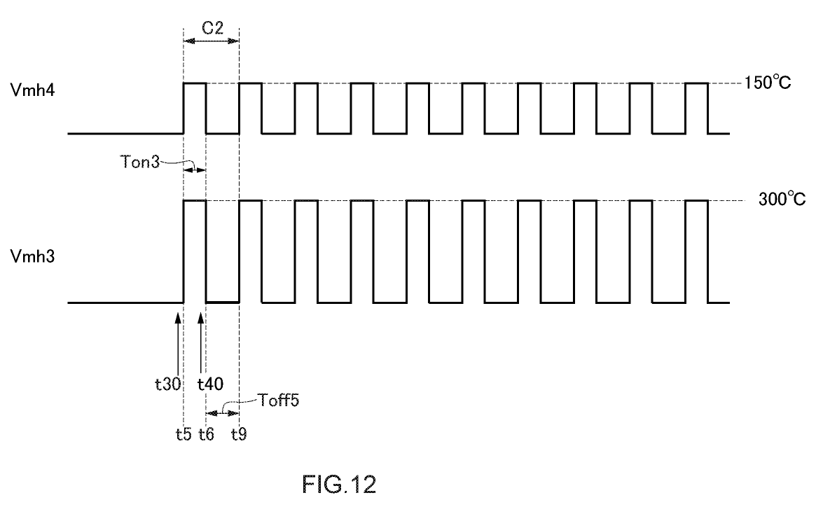

FIG. 11 is a flowchart for explaining the operation of the sensor part 21. FIG. 12 is a timing chart for explaining the operation of the sensor part 21.

In the gas concentration measurement operation using the sensor part 21, first, the signal processing circuit 40 included in the gas sensor 100 samples the temperature detection signal Vtemp and calculates the ambient temperature (step 201). The sampling of the temperature detection signal Vtemp is performed at timing t30 illustrated in FIG. 12. Timing t30 is immediately before timing t5, at which the thermistors Rd3 and Rd4 start being heated by the heaters MH3 and MH4, respectively.

Then, the control circuit 46 included in the signal processing circuit 40 calculates a heater command value based on the ambient temperature and outputs it to the D/A converter 45 to start heating the thermistors Rd3 and Rd4 (step 202). The heater command value is converted by the D/A converter 45 into heater voltages Vmh3 and Vmh4, which are applied to the heaters MH3 and MH4, respectively. In step 202, the thermistor Rd3 is heated to, for example, about 300°C, and the thermistor Rd4 is heated to, for example, about 150°C (when the concentration of CO₂ gas in the measurement atmosphere is, for example, zero, the thermistor Rd3 is heated to, for example, about 300°C, and the thermistor Rd4 is heated to, for example, about 150°C). The thermistors Rd3 and Rd4 start being heated at timing t5 illustrated in FIG. 12.

The thermistors Rd3 and Rd4 are not stable in terms of temperature until a predetermined time has elapsed from timing t5 at which they start being heated, so that a predetermined standby time is required until the detection signal Vgas34 is sampled after the start of heating. The signal processing circuit 40 samples the detection signal Vgas34 at timing t40 at which a predetermined standby time has elapsed (step 203). Subsequently, the signal processing circuit 40 calculates the output signal Vout from the detection signal Vgas34 and outputs the calculated output signal Vout to the outside.

Then, the control circuit 46 resets the heater command value to stop heating the thermistors Rd3 and Rd4 (step 204). The thermistors Rd3 and Rd4 stop being heated at timing t6 illustrated in FIG. 12. Thus, the gas concentration measurement operation using the sensor part 21 is completed. In the gas concentration measurement operation using the sensor part 21, the time interval between timing t5 at which the thermistors Rd3 and Rd4 start being heated and timing t6 at which they stop being heated is defined as an on-period Ton3. The on-period Ton3 may be set to have the same length as the on-period Ton1 of sensor part 11.

After the elapse of a predetermined off-period Toff5 from timing t6, the control circuit 46 resumes the gas concentration measurement operation. For example, the signal processing circuit 40 samples the temperature detection signal Vtemp and calculates the ambient temperature (step 201), and the control circuit 46 outputs the heater command value calculated based on the ambient temperature, to thereby start heating the thermistors Rd3 and Rd4 (step 202). The thermistors Rd3 and Rd4 start being heated at timing t9 illustrated in FIG. 12. Thus, the off-period Toff5 is defined as the time interval between timing t6 at which the thermistors Rd3 and Rd4 stop being heated and timing t9 at which the thermistors Rd3 and Rd4 start being heated again. The off-period Toff5 may be shorter in length than the off-period Toff2 set in the sensor part 11. The off-period Toff5 may be the same as or shorter than the off-period Toff1 set in the sensor part 11.

By repeatedly performing the above-described operation at predetermined intervals, the concentration of the target gas contained in the surrounding environment can be detected periodically. An execution cycle C2 of the gas concentration measurement operation using the sensor part 21 is defined as the period from time t5 to time t9. The execution cycle C2 may be set shorter than the execution cycle C1 so as to allow the measurement result to track concentration changes even when the CO₂ gas concentration undergoes rapid and significant changes. The execution cycle C2 is set to, for example, 2 seconds.

In the above example, the gas concentration measurement operation and the dummy heating operation are alternately performed using the sensor part 11, whereas the gas concentration measurement operations are continuously performed using the sensor part 21 with an off-period Toff5 interposed therebetween, without performing any dummy heating operation (the dummy heating operation refers to heating the thermistor Rd3 to, for example, around 150°C, referred to as the second temperature range, using the heater MH3 for a predetermined time, and to heating the thermistor Rd4 to, for example, around 300°C, referred to as the first temperature range, using the heater MH4 for a predetermined time).

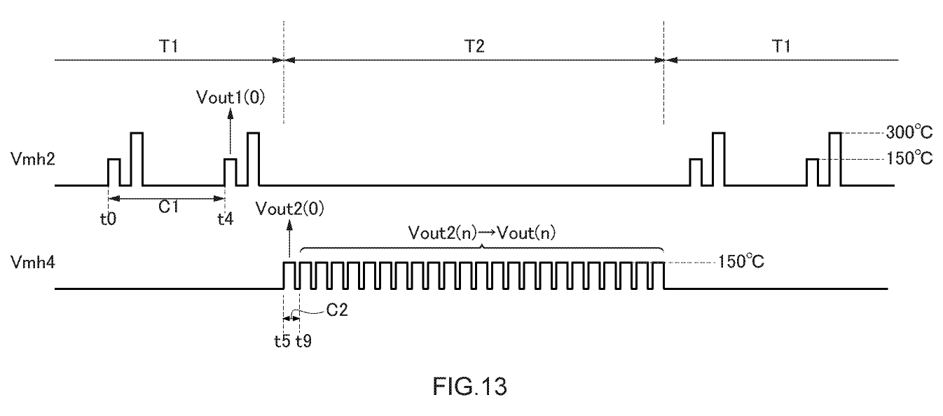

FIG. 13 is a timing chart for explaining an example of the operations of the sensor parts 11 and 21 in the operation periods T1 and T2 illustrated in FIG. 2A.

In the example illustrated in FIG. 13, the gas concentration measurement operation using the sensor part 21 in the operation period T2 is performed more frequently than that using the sensor part 11 in the operation period T1. The execution cycle C1 of the gas concentration measurement operation using the sensor part 11 in the operation period T1 is defined as the period from time t0 to time t4. The execution cycle C2 of the gas concentration measurement operation using the sensor part 21 in the operation period T2 is defined as the period from time t5 to time t9. The execution cycle C2 is shorter than the execution cycle C1 and, accordingly, the execution frequency per unit time of the gas concentration measurement operation using the sensor part 21 in the operation period T2 is higher than the execution frequency per unit time of the gas concentration measurement operation using the sensor part 11 in the operation period T1.

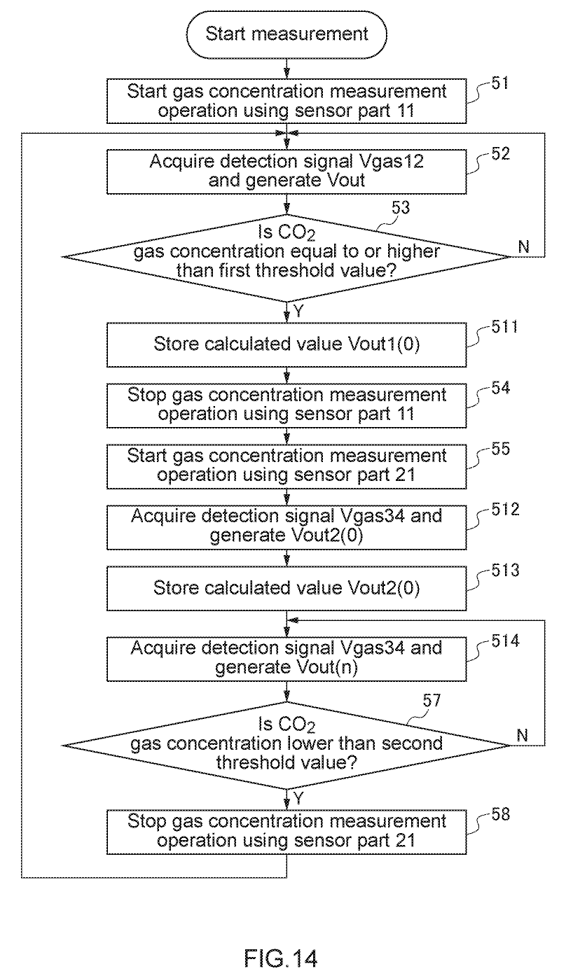

FIG. 14 is a flowchart for explaining a modification of the gas concentration measurement operation illustrated in FIG. 2A.

The modified operation illustrated in FIG. 14 differs from the operation illustrated in FIG. 3 in that steps 511 to 513 are added and that step 56 is replaced with step 514. Other basic operations are the same as those illustrated in FIG. 3, so the same reference numerals are given to the same steps, and overlapping description will be omitted.

Step 511 is a step in which the calculated value Vout1 (0) illustrated in FIG. 13 is stored in the control circuit 46. The calculated value Vout1(0) is the value of the output signal Vout when it is determined that the CO₂ gas concentration is higher than the first threshold value. Step 512 is, as illustrated in FIG. 13, a step in which the calculated value Vout2(0) is generated based on the first detection signal Vgas34 obtained after the start of the gas concentration measurement operation using the sensor part 21. The calculated value Vout2(0) corresponds to the value of the output signal Vout calculated based on the first detection signal Vgas34 obtained after the start of the gas concentration measurement operation using the sensor part 21. The calculated value Vout2(0) is stored in the control circuit 46 in step 513.

Step 514 is a step in which the output signal Vout(n) is generated based on the (n+1)-th detection signal Vgas34 (n is an integer equal to or greater than 0). The output signal Vout(n) refers to the output signal Vout generated based on the (n+1)-th detection signal Vgas34. In generating the output signal Vout(n) in step 514, not only the calculated value Vout2(n) based on the (n+1)-th detection signal Vgas34 but also the calculated values Vout1(0) and Vout2(0), which are stored in the control circuit 46, are used. For example, the output signal Vout(n) is calculated according to the following equation: Vout(n) = Vout1(0) + (Vout2(n) – Vout2(0)). That is, unlike step 56 of FIG. 3, in which the output signal Vout is generated directly based on the current detection signal Vgas34, the output signal Vout(n) is generated by adding, to the calculated value Vout1(0) generated based on the detection signal Vgas12 when the CO₂ concentration is determined to be equal to or higher than the first threshold value, a difference obtained by subtracting the calculated value Vout2(0) based on the first detection signal Vgas34 from the calculated value Vout2(n) based on the current (i.e., (n+1)-th) detection signal Vgas34. The above equation can be expressed as follows: Vout(n) = Vout2(n) + (Vout1(0) – Vout2(0)). Thus, to rephrase the above, the output signal Vout(n) is generated by adding a difference obtained by subtracting the calculated value Vout2(0) from the calculated value Vout1(0) to the calculated value Vout2(n) based on the current (i.e., (n+1)-th) detection signal Vgas34. That is, the output signal Vout(n) is generated by correcting the calculated value Vout2(n) through adding the difference obtained by subtracting the calculated value Vout2(0) from the calculated value Vout1(0).

Even if the sensor parts 11 and 21 perform the gas concentration measurement operation at the same time, the value of the output signal Vout based on the detection signal Vgas12 from the sensor part 11 and the value of the output signal Vout based on the detection signal Vgas34 from the sensor part 21 may differ due to characteristic differences between the sensor parts 11 and 21. The presence of characteristic differences between the sensor parts 11 and 21 may cause measurement errors in the gas concentration if the output signal Vout is generated directly based on the current detection signal Vgas34. However, when the output signal Vout(n) is generated as described above, such measurement errors can be reduced.

In the modification illustrated in FIG. 14, in step 57, it is determined whether the CO₂ gas concentration indicated by the output signal Vout generated based on the calculated value Vout2(n), the calculated value Vout1(0), and the calculated value Vout2(0) is lower than the second threshold value.

In the above modification, the calculated value Vout2(0) based on the detection signal Vgas34 immediately after the acquisition of the calculated value Vout1(0) is used to generate the output signal Vout(n). Instead of using this, the calculated value Vout2(1) based on the second detection signal Vgas34 after the acquisition of the calculated value Vout1(0) may be used. Thus, instead of using the calculated value Vout2(0) obtained in an unstable state immediately after the start-up of sensor part 21, the calculated value Vout2(1), obtained in a more stable state, is used. This allows the gas concentration to be measured more accurately.

Alternatively, instead of using the calculated value Vout2(0), the average of a plurality of calculated values Vout2(0), Vout2(1), Vout2(2), …, based on a plurality of detection signals Vgas34 obtained after the acquisition of the calculated value Vout1(0) may be used. This allows the gas concentration to be measured more accurately. In this case, the average may be calculated without using the calculated value Vout2(0).

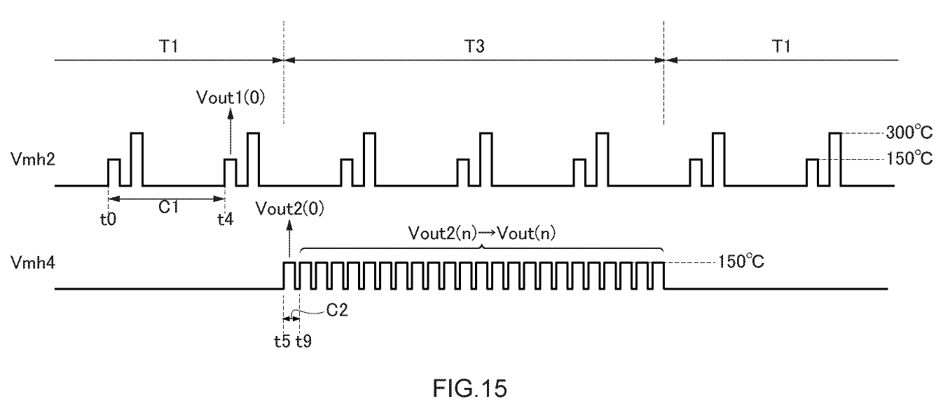

FIG. 15 is a timing chart for explaining an example of the operations of the sensor parts 11 and 21 in the operation periods T1 and T3 illustrated in FIG. 2B.

In the example illustrated in FIG. 15, the gas concentration measurement operation using the sensor part 21 in the operation period T3 is performed more frequently than that using the sensor part 11 in the operation periods T1 and T3. The execution cycle C1 of the gas concentration measurement operation using the sensor part 11 in the operation periods T1 and T3 is defined as the period from time t0 to time t4. The execution cycle C2 of the gas concentration measurement operation using the sensor part 21 in the operation period T3 is defined as the period from time t5 to time t9. The execution cycle C2 is shorter than the execution cycle C1 and, accordingly, the execution frequency per unit time of the gas concentration measurement operation using the sensor part 21 in the operation period T3 is higher than the execution frequency per unit time of the gas concentration measurement operation using the sensor part 11 in the operation periods T1 and T3.

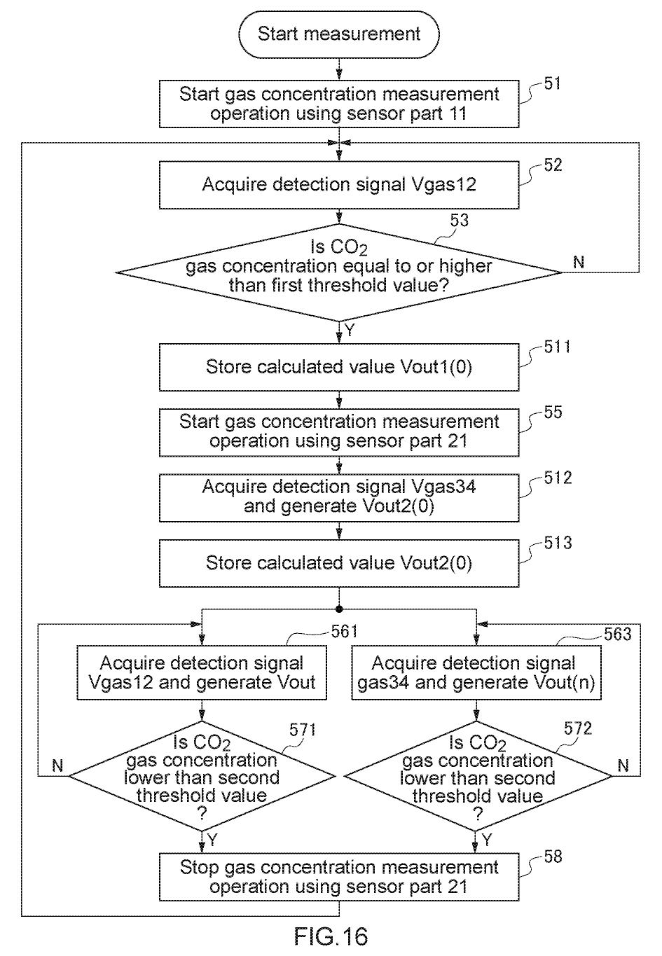

FIG. 16 is a flowchart for explaining a first modification of the gas concentration measurement operation illustrated in FIG. 2B.

The operation according to the first modification illustrated in FIG. 16 differs from the operation illustrated in FIG. 5 in that steps 511 to 513 are added and that step 562 is replaced with step 563. Other basic operations are the same as those illustrated in FIG. 5, so the same reference numerals are given to the same steps, and overlapping description will be omitted.

Step 511 is a step in which the calculated value Vout1 (0) illustrated in FIG. 15 is stored in the control circuit 46. Step 512 is, as illustrated in FIG. 15, a step in which the calculated value Vout2(0) is generated based on the first detection signal Vgas34 obtained after the start of the gas concentration measurement operation using the sensor part 21. The calculated value Vout2(0) is stored in the control circuit 46 in step 513.

Step 563 is a step in which the output signal Vout(n) is generated based on the (n+1)-th detection signal Vgas34. In generating the output signal Vout(n) in step 563, not only the calculated value Vout2(n) based on the (n+1)-th detection signal Vgas34 but also the calculated values Vout1(0) and Vout2(0), which are stored in the control circuit 46, are used. For example, the output signal Vout(n) is calculated according to the following equation: Vout(n) = Vout1(0) + (Vout2(n) – Vout2(0)). That is, unlike step 562 of FIG. 5, in which the output signal Vout is generated directly based on the current detection signal Vgas34, the output signal Vout(n) is generated by adding, to the calculated value Vout1(0) generated based on the detection signal Vgas12 when the CO₂ concentration is determined to be equal to or higher than the first threshold value, a difference obtained by subtracting the calculated value Vout2(0) based on the first detection signal Vgas34 from the calculated value Vout2(n) based on the current (i.e., (n+1)-th) detection signal Vgas34. The above equation can be expressed as follows: Vout(n) = Vout2(n) + (Vout1(0) – Vout2(0)). Thus, to rephrase the above, the output signal Vout(n)is generated by adding a difference obtained by subtracting the calculated value Vout2(0) from the calculated value Vout1(0) to the calculated value Vout2(n) based on the current (i.e., (n+1)-th) detection signal Vgas34. That is, the output signal Vout(n) is generated by correcting the calculated value Vout2(n) through adding the difference obtained by subtracting the calculated value Vout2(0) from the calculated value Vout1(0). Thus, even if characteristic differences exist between the sensor parts 11 and 21, measurement errors in the gas concentration can be reduced.

In the first modification illustrated in FIG. 16, in step 572, it is determined whether the CO₂ gas concentration indicated by the output signal Vout generated based on the calculated value Vout2(n), the calculated value Vout1(0), and the calculated value Vout2(0) is lower than the second threshold value.

In the above first modification, the calculated value Vout2(0) based on the detection signal Vgas34 immediately after the acquisition of the calculated value Vout1(0) is used to generate the output signal Vout(n). Instead of using this, the calculated value Vout2(1) based on the second detection signal Vgas34 after the acquisition of the calculated value Vout1(0) may be used. Alternatively, instead of using the calculated value Vout2(0), the average of a plurality of calculated values Vout2(0), Vout2(1), Vout2(2), …, based on a plurality of detection signals Vgas34 obtained after the acquisition of the calculated value Vout1(0) may be used. In this case, the average may be calculated without using the calculated value Vout2(0).

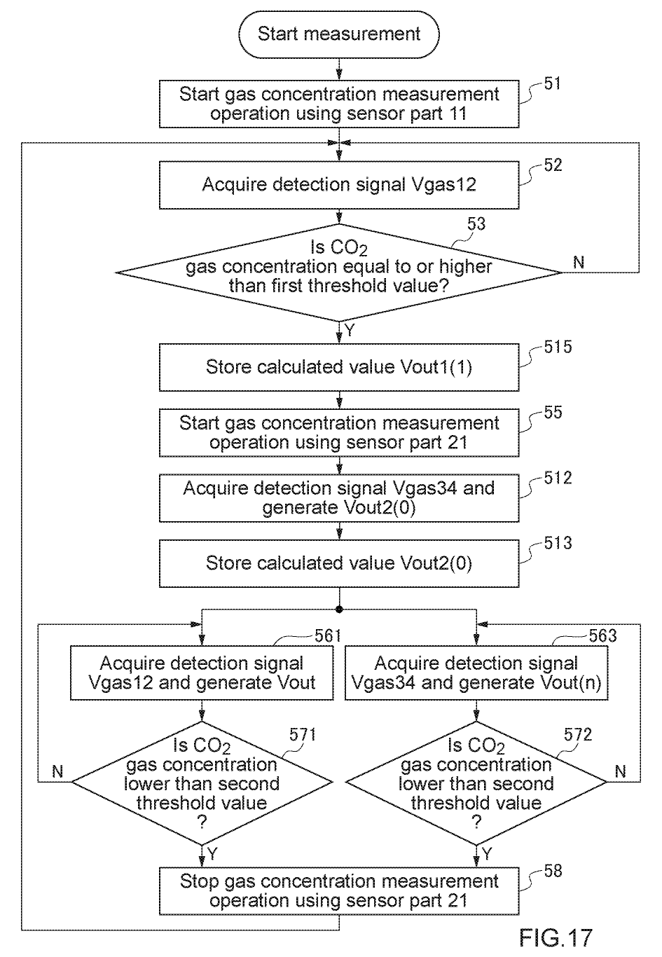

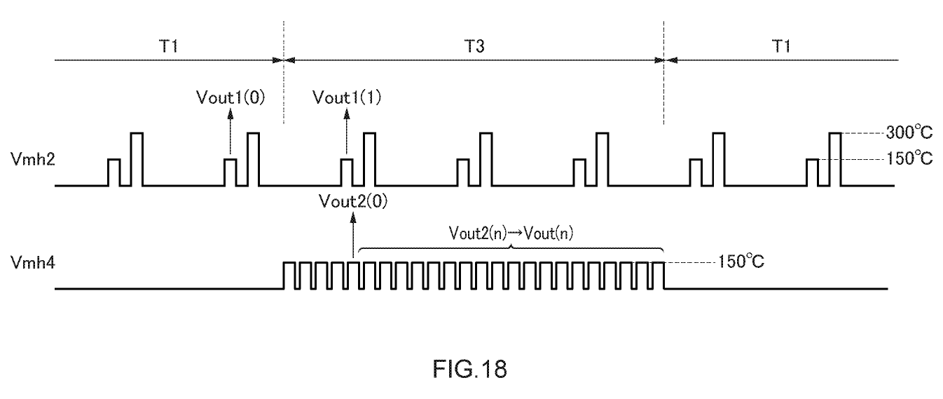

FIG. 17 is a flowchart for explaining a second modification of the gas concentration measurement operation illustrated in FIG. 2B. FIG. 18 is a timing chart for explaining the operations of the sensor parts 11 and 21 according to the second modification in the operation periods T1 and T3 illustrated in FIG. 2B.

The operation according to the second modification illustrated in FIG. 17 differs from that according to the first modification illustrated in FIG. 16 in that step 511 is replaced with step 515. Thus, the generation timing of the calculated value Vout2(0) in step 512 differs from that in the operation according to the first modification illustrated in FIG. 16. Other basic operations are the same as those according to the first modification illustrated in FIG. 16, so the same reference numerals are given to the same steps, and overlapping description will be omitted.

Step 515 is a step in which the calculated value Vout1(1) illustrated in FIG. 18 is stored in the control circuit 46. The calculated value Vout1(1) corresponds to the value of the output signal Vout that is calculated based on the detection signal Vgas12 obtained in the first gas concentration measurement operation using the sensor part 11 after it has been determined that the CO₂ gas concentration is equal to or higher than the first threshold value. In the second modification, when generating the output signal Vout(n) in step 563, not only the calculated value Vout2(n) based on the (n+1)-th detection signal Vgas34 obtained after the gas concentration measurement operation in which the sensor part 11 calculates the calculated value Vout 1(1) but also the calculated values Vout1(1) and Vout2(0), which are stored in the control circuit 46, are used. In the second modification illustrated in FIG. 17, the calculated value Vout2(0) is generated based on the first detection signal Vgas34 after the gas concentration measurement operation in which the sensor part 11 calculates the calculated value Vout1(1). For example, the output signal Vout(n) is calculated according to the following equation: Vout(n) = Vout1(1) + (Vout2(n) – Vout2(0)).

That is, in the second modification, as illustrated in the timing chart of FIG. 18, when it is determined that the CO₂ gas concentration is equal to or higher than the first threshold value, the calculated value Vout1(1), which is generated based on the detection signal Vgas12 obtained in the first gas concentration measurement operation performed using the sensor part 11 after this determination, is stored, instead of the calculated value Vout1(0) generated in the gas concentration measurement operation in which the determination has been made. Then, the output signal Vout(n) is generated by adding to the stored calculated value Vout1(1) a difference obtained by subtracting the calculated value Vout2(0), which is based on the first detection signal Vgas34 obtained after the gas concentration measurement operation in which the sensor part 11 calculates the calculated value Vout1(1), from the calculated value Vout2(n) based on the current detection signal Vgas34. In other words, instead of the calculated value Vout1(0) used in the first modification, the calculated value Vout1(1) is used to generate the output signal Vout(n). This makes it possible to reduce the difference between the acquisition timing of the calculated value Vout1(1) and that of the calculated value Vout2(0), thereby reducing measurement errors due to the timing difference.

In the second modification illustrated in FIG. 17, in step 572, it is determined whether the CO₂ gas concentration indicated by the output signal Vout generated based on the calculated value Vout2(n), the calculated value Vout1(1), and the calculated value Vout2(0) is lower than the second threshold value.

In the second modification, the calculated value Vout1(i) (i is an integer equal to or greater than 2) may be generated based on the detection signal Vgas12 obtained in the second and subsequent gas concentration measurement operations using the sensor part 11 after it has been determined that the CO₂ gas concentration is equal to or higher than the first threshold value and used instead of the calculated value Vout1(1). In this case, the Vout2(m) (m is an integer equal to or greater than 1) may be generated based on the detection signal Vgas34 obtained in the first gas concentration measurement operation using the sensor part 21 after the gas concentration measurement operation in which the sensor part 11 calculates the calculated value Vout1(i) and used instead of the calculated value Vout2(0).

In the above second modification, the calculated value Vout2(0) based on the detection signal Vgas34 immediately after the acquisition of the calculated value Vout1(1) is used to generate the output signal Vout(n). Instead of using this, the calculated value Vout2(1) based on the second detection signal Vgas34 after the acquisition of the calculated value Vout1(1) may be used. Alternatively, instead of using the calculated value Vout2(0), the average of a plurality of calculated values Vout2(0), Vout2(1), Vout2(2), …, based on a plurality of detection signals Vgas34 obtained after the acquisition of the calculated value Vout1(1) may be used. In this case, the average may be calculated without using the calculated value Vout2(0).

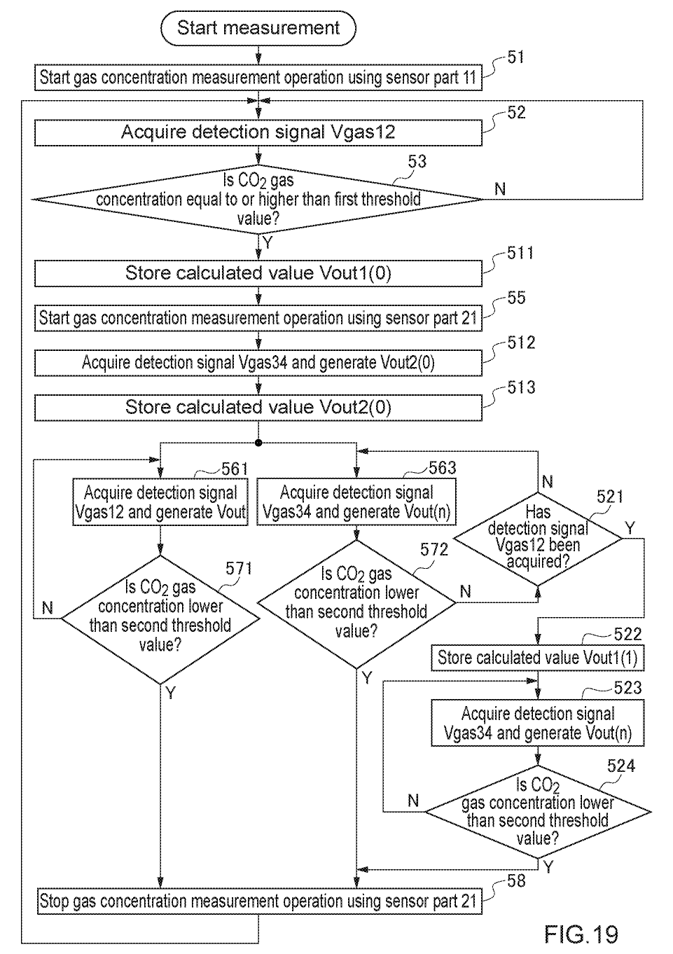

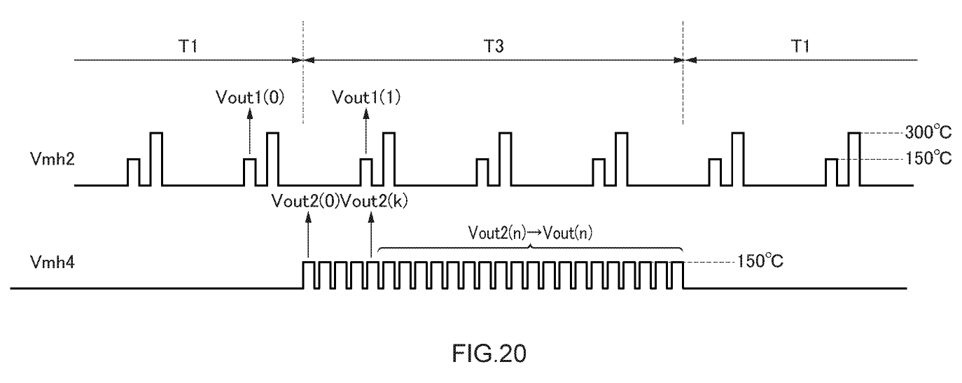

FIG. 19 is a flowchart for explaining a third modification of the gas concentration measurement operation illustrated in FIG. 2B. FIG. 20 is a timing chart for explaining the operations of the sensor parts 11 and 21 according to the third modification in the operation periods T1 and T3 illustrated in FIG. 2B.

The operation according to the third modification illustrated in FIG. 19 differs from that according to the first modification illustrated in FIG. 16 in that steps 521 to 524 are added. Other basic operations are the same as those according to the first modification illustrated in FIG. 16, so the same reference numerals are given to the same steps, and overlapping description will be omitted.

As in the first modification, in the third modification, when it is determined in step 53 that the CO₂ gas concentration is equal to or higher than the first threshold value, the output signal Vout(n) is generated using the calculated value Vout1(0), the calculated value Vout2(0), and the Vout2(n) during the period before the next gas concentration measurement operation using the sensor part 11 is performed.

Then, when the next gas concentration measurement operation using the sensor part 11 is performed (Y in step 521), the calculated value Vout1(1) is stored in the control circuit 46 (step 522). Thereafter, the calculated value Vout2(0) in the second modification is replaced with the calculated value Vout2(k) (k is an integer equal to or greater than 1), and the calculated value Vout1(1), the calculated value Vout2(k), and the calculated value Vout2(n) are used to generate the output signal Vout(n) (step 523). The calculated value Vout2(k) corresponds to the value of the output signal Vout calculated based on the detection signal Vgas34 obtained immediately after the first gas concentration measurement operation using the sensor part 11 after it has been determined that the CO₂ gas concentration is equal to or higher than the first threshold value in step 53.

Thereafter, it is determined whether the CO₂ gas concentration indicated by the output signal Vout generated based on the calculated value Vout2(n), the calculated value Vout1(1), and the calculated value Vout2(k) is lower than the second threshold value (step 524). When it is determined that the current CO₂ gas concentration is lower than the second threshold value, the gas concentration measurement operation using the sensor part 21 is stopped (step 58), and the processing flow returns to step 52.

That is, in the third modification, as illustrated in the timing chart of FIG. 20, when it is determined that the CO₂ gas concentration is equal to or higher than the first threshold value, the same operation as that according to the first modification is performed during the period before the k-th gas concentration measurement operation using the sensor part 21, and the same operation as that according to the second modification is performed from the (k+1)-th gas concentration measurement operation onward. The k-th gas concentration measurement operation using the sensor part 21 refers to the gas concentration measurement operation using the sensor part 21 that is performed immediately after the first gas concentration measurement operation using the sensor part 11 after it has been determined that the CO₂ gas concentration is equal to or higher than the first threshold value in step 53. This makes it possible to generate the output signal Vout(n) at an earlier stage, as in the first modification, while also reducing measurement errors, as in the second modification.

In the third modification, the calculated value Vout1(i) (i is an integer equal to or greater than 2) may be generated based on the detection signal Vgas12 obtained in the second and subsequent gas concentration measurement operations using the sensor part 11 after it has been determined that the CO₂ gas concentration is equal to or higher than the first threshold value and used instead of the calculated value Vout1(1). In this case, the Vout2(k) may be updated based on the detection signal Vgas34 obtained in the first gas concentration measurement operation using the sensor part 21 after the gas concentration measurement operation in which the sensor part 11 calculates the calculated value Vout1(i).

Further, in the third modification, the calculated value Vout2(0) based on the detection signal Vgas34 immediately after the acquisition of the calculated value Vout1(0) or the calculated value Vout2(k) based on the detection signal Vgas34 immediately after the acquisition of the calculated value Vout1(1) is used to generate the output signal Vout(n). Instead of using the above calculated value Vout2(0), the calculated value Vout2(1) based on the second detection signal Vgas34 after the acquisition of the calculated value Vout1(0) may be used. Further, instead of using the above calculated value Vout2(k), the calculated value Vout2(k+1) based on the second detection signal Vgas34 after the acquisition of the calculated value Vout1(1) may be used. Furthermore, the average of a plurality of calculated values Vout2(0), Vout2(1), Vout2(2), …, based on a plurality of detection signals Vgas34 obtained after the acquisition of the calculated value Vout1(0) may be used. In this case, the average may be calculated without using the calculated value Vout2(0). Furthermore, instead of using the calculated value Vout2(k), the average of a plurality of calculated values Vout2(k+1), Vout2(k+2), Vout2(k+3), …, based on a plurality of detection signals Vgas34 obtained after the acquisition of the calculated value Vout1(1) may be used. In this case, the average may be calculated without using the calculated value Vout2(k+1).

Although the dummy heating operation is not performed for the sensor part 21, it may be performed for the sensor part 21.

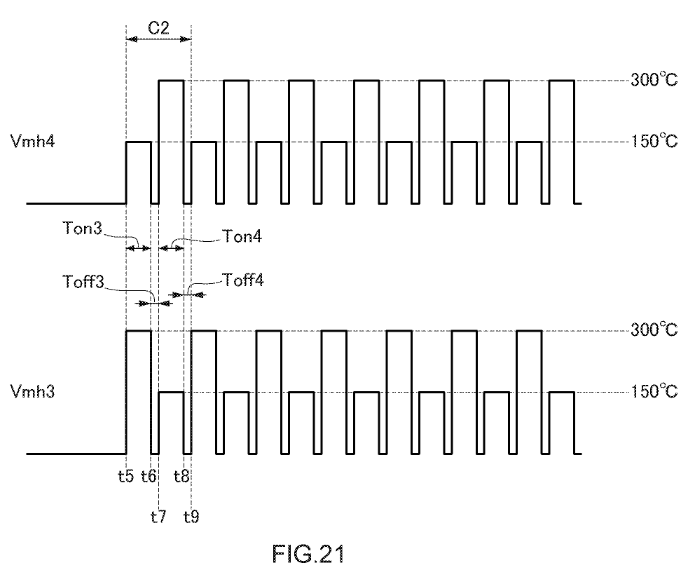

FIG. 21 is a timing chart for explaining the operation of the sensor part 21 according to a first modification.

As illustrated in FIG. 21, in the operation of the sensor part 21 according to the first modification, during the gas concentration measurement operation using the sensor part 21, the dummy heating is performed for the thermistors Rd3 and Rd4 during the interval between timing t6, at which heating of the thermistors Rd3 and Rd4 is stopped, and timing t9, at which heating of the thermistors Rd3 and Rd4 is resumed. In this operation, the thermistor Rd3 is heated to, for example, about 150°C (an example of the second temperature range), and the thermistor Rd4 is heated to, for example, about 300°C (an example of the first temperature range). The dummy heating of the thermistors Rd3 and Rd4 is started at timing t7 illustrated in FIG. 21. In the gas concentration measurement operation using the sensor part 21, the time interval between timing t6, at which the thermistors Rd3 and Rd4 stop being heated, and timing t7, at which their dummy heating starts, is defined as an off-period Toff3.

The dummy heating of the thermistors Rd3 and Rd4 is performed during the interval from timing t7 to timing t8, during which the predetermined on-period Ton4 elapses. The time interval between timing t8, at which the dummy heating of the thermistors Rd3 and Rd4 is stopped, and timing t9, at which the thermistors Rd3 and Rd4 start being heated is defined as an off-period Toff4.

Thus, the dummy heating may be performed for the thermistors Rd3 and Rd4 included in the sensor part 21. This reduces the thermal history difference between the thermistors Rd3 and Rd4 to suppress temporal changes in the sensor part 21 due to the thermal history difference. In order to further reduce the thermal history difference, the on-periods Ton3 and Ton4 may be set to the same length.

As described above, in the present embodiment, when the concentration of CO₂ gas, which is the target gas, is lower than the first threshold value, stable gas concentration measurement with reduced temporal changes can be performed using the sensor part 11. Further, when the CO₂ gas concentration is equal to or higher than the first threshold value, the output signal Vout can track concentration changes through high-frequency gas concentration measurements using the sensor part 21, even when the CO₂ gas concentration undergoes rapid and significant variations. In addition, when the CO₂ gas concentration as the detection target is lower than the first threshold value, the gas concentration measurement operation using the sensor part 21 is stopped, thereby reducing power consumption during periods in which the CO₂ gas concentration remains stable at low levels.

Further, in the operation period T3, the gas concentration measurement operation using the sensor part 11 and that using the sensor part 21 may be performed asynchronously or with their timings offset from each other. For example, when the gas concentration measurement operations using the sensor parts 11 and 21 are offset in timing to prevent overlapping of time t20 shown in FIG. 7 and time t40 shown in FIG. 12, the simultaneous input of the amplified signals Vamp1 and Vamp2 to the AD converter 44 can be prevented.

In the examples illustrated in FIGS. 11 to 20, the dummy heating operation is not performed while the gas concentration measurement operation is continuously performed using the sensor part 21. Accordingly, a thermal history difference occurs between the thermistors Rd3 and Rd4, resulting in temporal changes in the sensor part 21. For example, when the cumulative execution time of the gas concentration measurement operation using the sensor part 21 during the operation period T2 is controlled to be shorter than that of the gas concentration measurement operation using the sensor part 11 during the operation period T1, temporal changes in the sensor part 21 are suppressed. Although the gas concentration measurement operation using the sensor part 21 is stopped during the operation period T1, the gas concentration measurement operation and the dummy heating operation using the sensor part 11 are periodically repeated, allowing the output signal Vout to be obtained at regular intervals. In order to suppress temporal changes in the sensor part 21, the first and second threshold values may be set such that the operation period T2 becomes shorter than the operation period T1 illustrated in FIG. 2. Similarly, when the cumulative execution time of the gas concentration measurement operation using the sensor part 21 during the operation period T3 is controlled to be shorter than that of the gas concentration measurement operation using the sensor part 11 during the operation periods T1 and T3, temporal changes in the sensor part 21 are suppressed.

Alternatively, during the operation period T3, the value of the output signal Vout calculated from the detection signal Vgas12 may be compared with that calculated from the detection signal Vgas34. When a non-negligible difference occurs between them, it is determined that the sensor part 21 experiences drift in the output signal Vout due to the influence of residual heat associated with the short execution cycle C2. In such a case, the first and second threshold values may be adjusted so that the difference becomes negligible. Further alternatively, when it is determined that the thermal history difference between the thermistors Rd3 and Rd4 has become sufficiently large to cause significant temporal changes in the sensor part 21, the gas concentration measurement operation using the sensor part 21 may be inhibited. Further, drift components and temporal change components may be removed by applying correction to the formula for calculating the output signal Vout from the detection signal Vgas34 based on the difference between the value of the output signal Vout calculated from Vgas12 and that calculated from Vgas34.

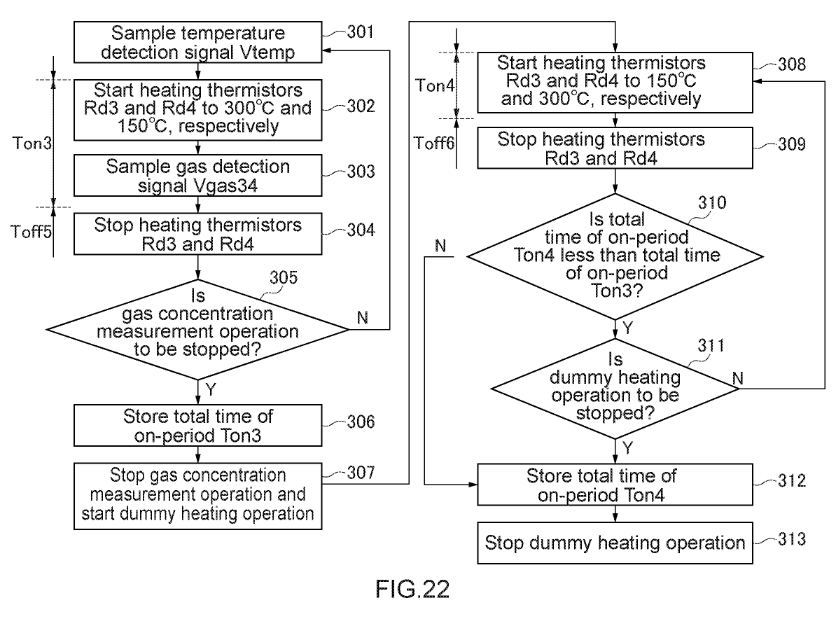

FIG. 22 is a flowchart for explaining the operation of the sensor part 21 according to a second modification.

As in the first modification, in the second modification illustrated in FIG. 22 as well, not only the gas concentration measurement operation but also the dummy heating operation is performed using the sensor part 21. In the gas concentration measurement operation using the sensor part 21, first, the signal processing circuit 40 included in the gas sensor 100 samples the temperature detection signal Vtemp and calculates the ambient temperature (step 301). Then, the control circuit 46 included in the signal processing circuit 40 calculates a heater command value based on the ambient temperature and outputs it to the D/A converter 45 to start heating the thermistors Rd3 and Rd4 (step 302). The heater command value is converted by the D/A converter 45 into heater voltages Vmh3 and Vmh4, which are applied to the heaters MH3 and MH4, respectively. In step 302, the thermistor Rd3 is heated to, for example, about 300°C, and the thermistor Rd4 is heated to, for example, about 150°C. (when the concentration of CO₂ gas in the measurement atmosphere is, for example, zero, the thermistor Rd3 is heated to, for example, about 300°C, and the thermistor Rd4 is heated to, for example, about 150°C).

The signal processing circuit 40 samples the detection signal Vgas34 at a timing at which a predetermined standby time has elapsed (step 303). Subsequently, the signal processing circuit 40 calculates the output signal Vout from the detection signal Vgas34 and outputs the calculated output signal Vout to the outside. Then, the control circuit 46 resets the heater command value to stop heating the thermistors Rd3 and Rd4 (step 304). In the gas concentration measurement operation using the sensor part 21, the time interval between the start of heating of the thermistors Rd3 and Rd4 in step 302 and the stop of their heating in step 304 is defined as an on-period Ton3.

Then, the control circuit 46 determines whether the gas concentration measurement operation using the sensor part 21 is to be stopped (step 305). The determination is made based on whether the CO₂ concentration measured by at least one of the sensor parts 11 and 21 is lower than the second threshold value. This determination may be made under the condition that both CO₂ concentrations measured by the sensor parts 11 and 21 are lower than the second threshold value, or under the condition that either one of them is lower than the second threshold value. When it is determined that the gas concentration measurement operation using the sensor part 21 is not to be stopped, the processing flow returns to step 301, in which the ambient temperature is calculated, and thereafter the thermistors Rd3 and Rd4 are heated again. In the gas concentration measurement operation using the sensor part 21, the time interval between the stop of heating of the thermistors Rd3 and Rd4 and the start of their heating is defined as the off-period Toff5. On the other hand, when it is determined that the gas concentration measurement operation using the sensor part 21 is to be stopped, the control circuit 46 stores the heating time (the total time of the on-period Ton3) of the thermistors Rd3 and Rd4 during the gas concentration measurement operation using the sensor part 21 (step 306), stops the gas concentration measurement operation using the sensor part 21, and starts the dummy heating operation for the sensor part 21 (step 307).

In the dummy heating operation for the sensor part 21, first, the heating of the thermistors Rd3 and Rd4 is started (step 308). For example, the thermistor Rd3 is heated to, for example, about 150°C, and the thermistor Rd4 is heated to, for example, about 300°C. Then, the control circuit 46 stops the heating of the thermistors Rd3 and Rd4 (step 309). In the dummy heating for the sensor part 21, the time interval between the start of heating of the thermistors Rd3 and Rd4 in step 308 and the stop of their heating in step 309 is defined as the on-period Ton4. The length of the on-period Ton4 may be the same as that of the on-period Ton3.

Then, the control circuit 46 determines whether the heating time (the total time of the on-period Ton4) of the thermistors Rd3 and Rd4 during the dummy heating operation for the sensor part 21 is less than the heating time (the total time of the on-period Ton3) of the thermistors Rd3 and Rd4 during the gas concentration measurement operation using the sensor part 21 (step 310). When it is determined that the total time of the on-period Ton4 is less than the total time of the on-period Ton3, that is, when it is determined that the total heating time during the dummy heating operation for the sensor part 21 is less than the total heating time during the gas concentration measurement operation using the sensor part 21, the control circuit 46 determines whether the dummy heating operation for the sensor part 21 is to be stopped (step 311). Whether the dummy heating operation for the sensor part 21 is to be stopped may be determined based on whether the CO₂ gas concentration measured using the sensor part 11 is equal to or higher than the first threshold value. Then, it is determined that the dummy heating operation for the sensor part 21 is not to be stopped, the processing flow returns to step 308, in which the thermistors Rd3 and Rd4 are heated again. In the dummy heating operation for the sensor part 21, the time interval between the stop of heating of the thermistors Rd3 and Rd4 and the start of their heating is defined as an off-period Toff6.

On the other hand, when it is determined in step 310 that the total time of the on-period Ton4 is equal to or greater than the on-period Ton3, that is, when it is determined that the total heating time during the dummy heating operation for the sensor part 21 is equal to or greater than the total heating time during the gas concentration measurement operation using the sensor part 21, or when it is determined in step 311 that the dummy heating operation for the sensor part 21 is to be stopped, the control circuit 46 stores the total heating time (the total time of the on-period Ton4) of the thermistors Rd3 and Rd4 during the dummy heating operation for the sensor part 21 (step 312), and then stops the dummy heating operation for the sensor part 21.

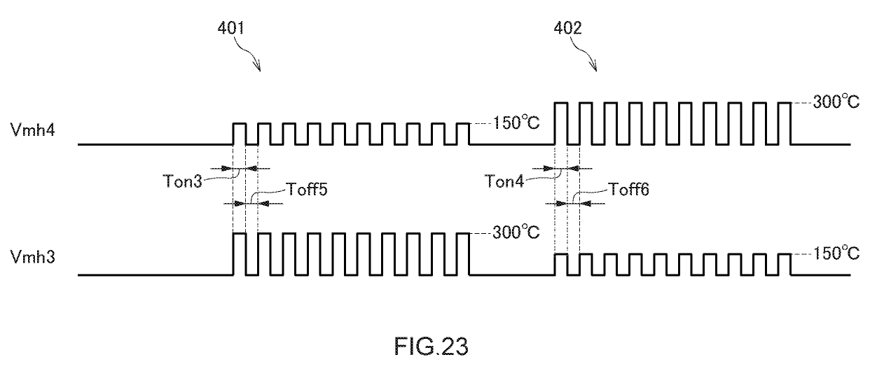

FIG. 23 is a timing chart for explaining an example of the operation of the sensor part 21 according to the second modification.

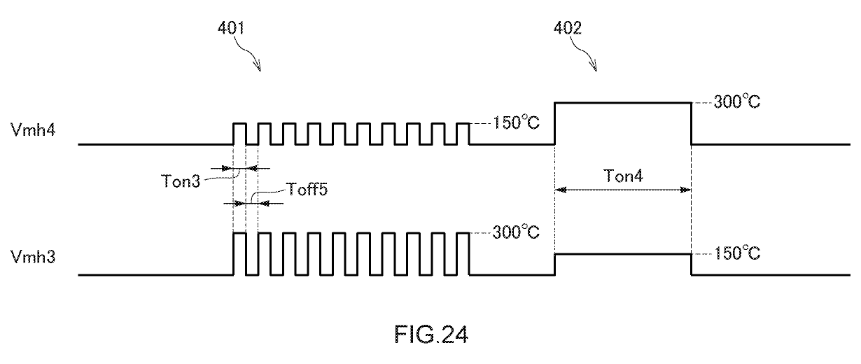

In the example illustrated in FIG. 23, the gas concentration measurement operation using the sensor part 21 is performed in the period 401, and then the dummy heating operation for the sensor part 21 is performed in the period 402. The period 401 starts upon determination that the CO₂ gas concentration measured using the sensor part 11 is equal to or higher than the first threshold value and ends upon determination that the CO₂ gas concentration measured using at least one of the sensor parts 11 and 21 is lower than the second threshold value. On the other hand, the period 402 may be started at a timing at which a predetermined standby time has elapsed after the end of the period 401. The lengths of the on-periods Ton3 and Ton4 need not be the same; the length of the on-period Ton4 may be longer than that of the on-period Ton3, as in the example illustrated in FIG. 24. In this case, step 308 may continue until the total heating time (the total time of the on-period Ton4) during the dummy heating operation for the sensor part 21 reaches the total heating time (total time of the on-period Ton3) during the gas concentration measurement operation using the sensor part 21.

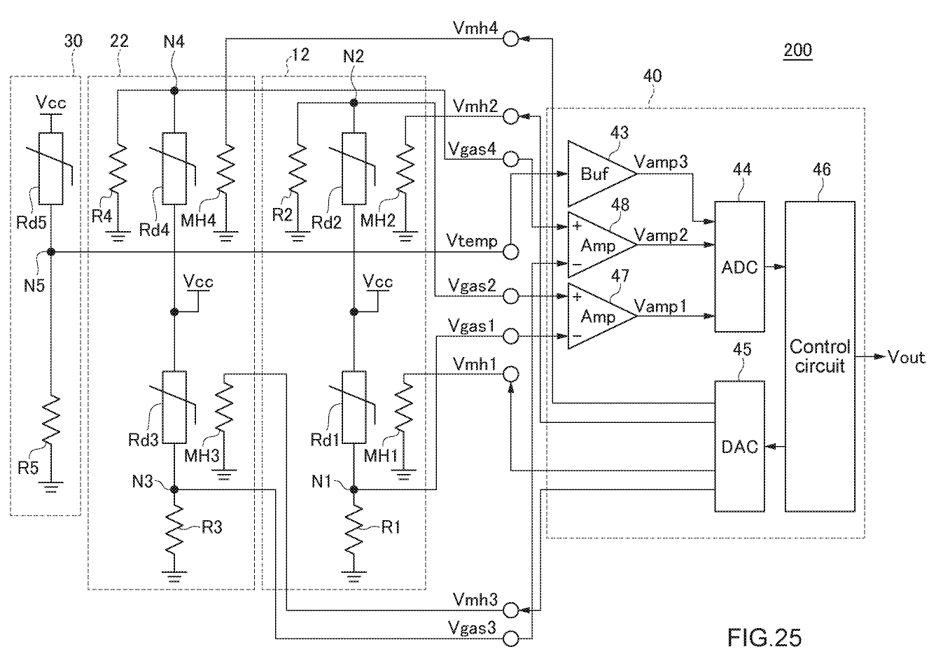

FIG. 25 is a circuit diagram illustrating the configuration of a gas sensor 200 according to a second embodiment of the technology described herein.

As illustrated in FIG. 25, the gas sensor 200 according to the second embodiment differs from the gas sensor 100 according to the first embodiment in the following points: the sensor part 11 is replaced with a sensor part 12; the sensor part 21 is replaced with a sensor part 22; and the differential amplifiers 41 and 42 included in the signal processing circuit 40 are replaced with differential amplifiers 47 and 48, respectively. Other basic configurations are the same as those of the gas sensor 100 according to the first embodiment, so the same reference numerals are given to the same elements, and overlapping description will be omitted.

The sensor part 12 includes the thermistor Rd1 and the fixed resistor R1, which are connected in series in this order between the power supply Vcc and the ground GND, the thermistor Rd2 and the fixed resistor R2, which are connected in series in this order between the power supply Vcc and the ground GND, and the heaters MH1 and MH2. The thermistor Rd1 changes in temperature in response to changes in the temperature of the heater MH1, and the thermistor Rd2 changes in temperature in response to changes in the temperature of the heater MH2. A detection signal Vgas1 appears at a node N1 between the thermistor Rd1 and the fixed resistor R1. A detection signal Vgas2 appears at a node N2 between the thermistor Rd2 and the fixed resistor R2.

The sensor part 22 includes the thermistor Rd3 and a fixed resistor R3, which are connected in series in this order between the power supply Vcc and the ground GND, the thermistor Rd4 and a fixed resistor R4, which are connected in series in this order between the power supply Vcc and the ground GND, and the heaters MH3 and MH4. The thermistor Rd3 changes in temperature in response to changes in the temperature of the heater MH3, and the thermistor Rd4 changes in temperature in response to changes in the temperature of the heater MH4. A detection signal Vgas3 appears at a node N3 between the thermistor Rd3 and the fixed resistor R3. A detection signal Vgas4 appears at a node N4 between the thermistor Rd4 and the fixed resistor R4.

The differential amplifier 47 compares the detection signals Vgas1 and Vgas2 to generate the amplified signal Vamp1 which is a signal obtained by amplifying a difference (= Vgas2 – Vgas1) in level between the detection signals Vgas1 and Vgas2. The differential amplifier 48 compares the detection signals Vgas3 and Vgas4 to generate the amplified signal Vamp2 which is a signal obtained by amplifying a difference (= Vgas4 – Vgas3) in level between the detection signals Vgas3 and Vgas4.

As exemplified by the gas sensor 200 according to the second embodiment, it is not essential that the thermistors Rd1 and Rd2 are connected in series; they may be connected in parallel between the power supply Vcc and the ground GND, and the amplified signal Vamp1 may be generated based on the difference between the output voltage (detection signal Vgas1) derived from the thermistor Rd1 and the output voltage (detection signal Vgas2) derived from the thermistor Rd2. Similarly, it is not essential that the thermistors Rd3 and Rd4 are connected in series; they may be connected in parallel between the power supply Vcc and the ground GND, and the amplified signal Vamp2 may be generated based on the difference between the output voltage (detection signal Vgas3) derived from the thermistor Rd3 and the output voltage (detection signal Vgas4) derived from the thermistor Rd4.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although thermistors serving as resistors are used as the temperature-sensing elements of the sensor parts 11, 12, 21, and 22 in the above embodiment, the present invention is not limited thereto; platinum (Pt) or tungsten (W) as resistors may also be used as the temperature-sensing elements.

Further, although the dummy heating is performed for the sensor part 11, it may be omitted when the influence of temporal changes is reduced.

The technology according to the present disclosure includes the following configuration examples, but not limited thereto.

A gas sensor according to an aspect of the present disclosure includes: a first sensor part configured to output a first detection signal according to a concentration of a target gas; a second sensor part configured to output a second detection signal according to the concentration of the target gas; and a signal processing circuit configured to control the first and second sensor parts and calculate the concentration of the target gas based on the first and second detection signals. The signal processing circuit is configured to control the first sensor part to repeatedly perform a first gas concentration measurement operation to acquire the first detection signal, control the second sensor part to start and repeatedly perform a second gas concentration measurement operation to acquire the second detection signal in response to the gas concentration calculated from the first detection signal reaching or exceeding a first threshold value, and stop the second gas concentration measurement operation in response to at least one of the gas concentration calculated from the first detection signal and the gas concentration calculated from at least the second detection signal reaching or falling below a second threshold value. An execution frequency per unit time of the second gas concentration measurement operation from its start to its stop is higher than an execution frequency per unit time of the first gas concentration measurement operation during the period in which the first gas concentration measurement operation is repeatedly performed. With the above configuration, the measurement results can track rapid and significant changes in the concentration of the target gas, while power consumption is reduced.

In the above gas sensor, the first sensor part may include first and second temperature-sensing elements, a first heater configured to heat the first temperature-sensing element, and a second heater configured to heat the second temperature-sensing element. The second sensor part may include third and fourth temperature-sensing elements, a third heater configured to heat the third temperature-sensing element, and a fourth heater configured to heat the fourth temperature-sensing element. When controlling the first sensor part, the signal processing circuit may be configured to alternately and repeatedly perform: the first gas concentration measurement operation, in which the first temperature-sensing element is heated to a first temperature range using the first heater, and the second temperature-sensing element is heated to a second temperature range using the second heater; and a first dummy heating operation, in which the first temperature-sensing element is heated to the second temperature range using the first heater, and the second temperature-sensing element is heated to the first temperature range using the second heater. When controlling the second sensor part, the signal processing circuit may be configured to repeatedly perform the second gas concentration measurement operation, in which the third temperature-sensing element is heated to the first temperature range using the third heater, and the fourth temperature-sensing element is heated to the second temperature range using the fourth heater. With the above configuration, temporal changes in the first sensor part can be suppressed.

In the above gas sensor, the first temperature range may be higher than the second temperature range, and a time interval from heating the first temperature-sensing element to the first temperature range using the first heater during the first gas concentration measurement operation to heating the first temperature-sensing element to the second temperature range using the first heater during the first dummy heating operation may be longer than a time interval from heating the second temperature-sensing element to the second temperature range using the second heater during the first gas concentration measurement operation to heating the second temperature-sensing element to the first temperature range using the second heater during the first dummy heating operation. With the above configuration, the thermal history difference between the first and second temperature-sensing elements can be reduced.

In the above gas sensor, an interval from the first dummy heating operation to the first gas concentration measurement operation may be ten times or more the execution time of the dummy heating operation. This makes it possible to reduce measurement errors in the first sensor part caused by residual heat.

In the above gas sensor, when controlling the second sensor part, the signal processing circuit may be configured to repeatedly perform the second gas concentration measurement operation without performing a heating operation in which the third temperature-sensing element is heated to the second temperature range using the third heater and the fourth temperature-sensing element is heated to the first temperature range using the fourth heater. This allows the second gas concentration measurement operation to be performed at a high frequency.

In the above gas sensor, when controlling the second sensor part, the signal processing circuit may be configured to further perform a second dummy heating operation, in which the third temperature-sensing element is heated to the second temperature range using the third heater, and the fourth temperature-sensing element is heated to the first temperature range using the fourth heater. This makes it possible to suppress temporal changes in the second sensor part. In this case, when controlling the second sensor part, the signal processing circuit may be configured to repeatedly and alternately perform the second gas concentration measurement operation and the second dummy heating operation. This facilitates the execution of control operations for the second sensor part.

In the above gas sensor, the first temperature-sensing element and the second temperature-sensing element may be connected in series, the third temperature-sensing element and the fourth temperature-sensing element may be connected in series, and the signal processing circuit may be configured to acquire the first detection signal from a node between the first temperature-sensing element and the second temperature-sensing element during the first gas concentration measurement operation, and may acquire the second detection signal from a node between the third temperature-sensing element and the fourth temperature-sensing element during the second gas concentration measurement operation. This makes it possible to simplify the circuit configurations of the first and second sensor parts.

In the above gas sensor, the signal processing circuit may be configured to perform the first gas concentration measurement operation and the second gas concentration measurement operation in parallel. This allows the concentration of the target gas to be measured through the first gas concentration measurement operation even while the second gas concentration measurement operation is being performed.

In the above gas sensor, a cumulative execution time of the second gas concentration measurement operation may be shorter than a cumulative execution time of the first gas concentration measurement operation. This makes it possible to suppress temporal changes in the second sensor part.

In the above gas sensor, during the second gas concentration measurement operation, the signal processing circuit may be configured to calculate the concentration of the target gas based on the second detection signal and the first detection signal. This makes it possible to reduce measurement errors even when characteristic differences occur between the first and second sensor parts.

In the above gas sensor, during the second gas concentration measurement operation, the signal processing circuit may be configured to calculate the concentration of the target gas by adding, to a first detection value based on the first detection signal, a difference obtained by subtracting a second detection value based on the second detection signal acquired after the first detection value from a third detection value based on a latest second detection signal.

What is claimed is:

1. A gas sensor comprising:
a first sensor part configured to output a first detection signal according to a concentration of a target gas;
a second sensor part configured to output a second detection signal according to the concentration of the target gas; and
a signal processing circuit configured to control the first and second sensor parts and calculate the concentration of the target gas based on the first and second detection signals,
wherein the signal processing circuit is configured to:
control the first sensor part to repeatedly perform a first gas concentration measurement operation to acquire the first detection signal;
control the second sensor part to start and repeatedly perform a second gas concentration measurement operation to acquire the second detection signal in response to a gas concentration calculated from the first detection signal reaching or exceeding a first threshold value; and
stop the second gas concentration measurement operation in response to at least one of the gas concentration calculated from the first detection signal and the gas concentration calculated from at least the second detection signal reaching or falling below a second threshold value, and
wherein an execution frequency per unit time of the second gas concentration measurement operation from its start to its stop is higher than an execution frequency per unit time of the first gas concentration measurement operation during a period in which the first gas concentration measurement operation is repeatedly performed.

2. The gas sensor as claimed in claim 1,
wherein the first sensor part includes first and second temperature-sensing elements, a first heater configured to heat the first temperature-sensing element, and a second heater configured to heat the second temperature-sensing element,
wherein the second sensor part includes third and fourth temperature-sensing elements, a third heater configured to heat the third temperature-sensing element, and a fourth heater configured to heat the fourth temperature-sensing element,
wherein, when controlling the first sensor part, the signal processing circuit is configured to alternately and repeatedly perform:
the first gas concentration measurement operation, in which the first temperature-sensing element is heated to a first temperature range using the first heater, and the second temperature-sensing element is heated to a second temperature range using the second heater; and
a first dummy heating operation, in which the first temperature-sensing element is heated to the second temperature range using the first heater, and the second temperature-sensing element is heated to the first temperature range using the second heater, and
wherein, when controlling the second sensor part, the signal processing circuit is configured to repeatedly perform the second gas concentration measurement operation, in which the third temperature-sensing element is heated to the first temperature range using the third heater, and the fourth temperature-sensing element is heated to the second temperature range using the fourth heater.

3. The gas sensor as claimed in claim 1,
wherein the first temperature range is higher than the second temperature range, and
wherein a time interval from heating the first temperature-sensing element to the first temperature range using the first heater during the first gas concentration measurement operation to heating the first temperature-sensing element to the second temperature range using the first heater during the first dummy heating operation is longer than a time interval from heating the second temperature-sensing element to the second temperature range using the second heater during the first gas concentration measurement operation to heating the second temperature-sensing element to the first temperature range using the second heater during the first dummy heating operation.

4. The gas sensor as claimed in claim 2, wherein an interval from the first dummy heating operation to the first gas concentration measurement operation is ten times or more an execution time of the dummy heating operation.

5. The gas sensor as claimed in claim 2, wherein, when controlling the second sensor part, the signal processing circuit is configured to repeatedly perform the second gas concentration measurement operation without performing a heating operation in which the third temperature-sensing element is heated to the second temperature range using the third heater and the fourth temperature-sensing element is heated to the first temperature range using the fourth heater.

6. The gas sensor as claimed in claim 2, wherein, when controlling the second sensor part, the signal processing circuit is configured to further perform a second dummy heating operation, in which the third temperature-sensing element is heated to the second temperature range using the third heater, and the fourth temperature-sensing element is heated to the first temperature range using the fourth heater.

7. The gas sensor as claimed in claim 6, wherein, when controlling the second sensor part, the signal processing circuit is configured to repeatedly and alternately perform the second gas concentration measurement operation and the second dummy heating operation.

8. The gas sensor as claimed in claim 2,
wherein the first temperature-sensing element and the second temperature-sensing element are connected in series,
wherein the third temperature-sensing element and the fourth temperature-sensing element are connected in series, and
wherein the signal processing circuit is configured to acquire the first detection signal from a node between the first temperature-sensing element and the second temperature-sensing element during the first gas concentration measurement operation, and acquire the second detection signal from a node between the third temperature-sensing element and the fourth temperature-sensing element during the second gas concentration measurement operation.

9. The gas sensor as claimed in claim 1, wherein the signal processing circuit is configured to perform the first gas concentration measurement operation and the second gas concentration measurement operation in parallel.

10. The gas sensor as claimed in claim 1, wherein a cumulative execution time of the second gas concentration measurement operation is shorter than a cumulative execution time of the first gas concentration measurement operation.

11. The gas sensor as claimed in claim 1, wherein, during the second gas concentration measurement operation, the signal processing circuit is configured to calculate the concentration of the target gas based on the second detection signal and the first detection signal.

12. The gas sensor as claimed in claim 11, wherein, during the second gas concentration measurement operation, the signal processing circuit is configured to calculate the concentration of the target gas by adding, to a first detection value based on the first detection signal, a difference obtained by subtracting a second detection value based on the second detection signal acquired after the first detection value from a third detection value based on a latest second detection signal.
